(19) United States
(12) Patent Application Publication
(10) Pub. No.: US 2011/0253138 A1
(43) Pub. Date: Oct. 20, 2011

(54) INHALER WITH AUDIBLE INDICATOR MEANS

(75) Inventors: John Briant, Hertfordshire (GB); Patrick Campbell, Hertfordshire (GB); Charles Cooke, Hertfordshire (GB); Christopher Groombridge, Hertfordshire (GB); James Daniel John, Hertfordshire (GB); William Bakewell, Hertfordshire (GB); Nicholas Harrison, Cambridgeshire (GB)

(73) Assignee: ASTRAZENECA AB, Sodertalje (SE)

(21) Appl. No.: 13/123,105

(22) PCT Filed: Oct. 7, 2009
(86) PCT No.: PCT/SE2009/051114

§ 371 Date: Jul. 11, 2011

Related U.S. Application Data
(60) Provisional application No. 61/103,600, filed on Oct. 8, 2008.

(51) Int. Cl. A61M 15/00

(52) U.S. Cl. 128/203.12

(57) ABSTRACT

The disclosure relates to an inhaler comprising an outlet through which doses of medicament may be dispensed. The inhaler also comprises a dispensing mechanism having a primed state, in which it is ready for dispensing a dose, and a fired state, in which it has dispensed a dose. In one aspect, the inhaler also comprises a disabler which, after the final dose has been dispensed, is activated to disable the dispensing mechanism from generating a sound which has been audible when the dispensing mechanism has moved from its primed state to its fired state. In a second aspect, the disabler prevents the dispensing mechanism from being in its primed state when an outlet cover is in an open position, whereby a not ready indication is presented to the user.

### TECHNICAL FIELD

The present invention relates to an inhaler, such as a dry powder inhaler, comprising an outlet, such as a mouthpiece or a nasal adapter, through which doses of medicament may be dispensed.

### BACKGROUND OF THE INVENTION

There are different types of inhalers on the market. A pressurized Metered Dose Inhaler (pMDI) releases a fixed dose of substance in aerosol form. A powder inhaler generally releases a dose of powdered substance entrained in an air stream. In a powder inhaler the powder may be provided in a bulk container of the inhaler from which doses of powder are metered for dispensing. As an alternative to a bulk container, powder inhalers may comprise a single compartment or a plurality of compartments for containing one or is more discrete doses of powdered substance. Such compartments may take the form of sealed blisters in a blister pack, a cavities-containing strip joined to a sealing strip or other suitable forms.

EP 1 220 698 discloses various embodiments of an inhaler for medicament in powder form. In the embodiment shown in FIGS. 10-14, the medicament is arranged in blisters on a rotatable disk inside the inhaler. When the airflow in the inhaler reaches a certain threshold value, a breath-activated activating means causes an elongated hollow body to pierce a blister so that the medicament is accessed. The breath-activated activating means comprises a pressure spring which urges a pressure member towards the blister. Such an inhaler could probably be provided with a dose counter to inform the patient of the number of doses dispensed from the inhaler or the number of doses remaining in the inhaler. However, even though such a dose counter would theoretically inform the user that all the doses have been dispensed, the user may be inadvertent and overlook the fact. Thus, the next time the user will inhale, an empty blister would be in the dispensing position and the patient might wrongly believe that he/she has inhaled a dose of medicament.

WO2009/008001A2 discloses a dry powder inhaler having a breath actuation feature. Opening a mouthpiece cap energises a spring. On inhalation, a flap is moved which triggers the release of the spring, driving a mechanism (i) to puncture a foil sealed medicament cavity and (ii) to ratchet an indexer around a set of ratchet teeth on the periphery of a disc of medicament cavities. On closing the cap, the mechanism is re-set, simultaneously resetting the breath flap and indexing the disc around by one cavity. The disc is moved around progressively as the cap is closed. The exact operation of the device is not fully explained in WO2009/008001A2, however it appears that the flap is reset substantially simultaneously with the disc reaching its final indexed position.

As the disc approaches its final cavity a ratchet wheel is driven round and blocks the disc from further movement after the last cavity is brought into registry with the inhalation path. It is not explained in WO2009/008001A2 what happens when a user tries to close the cap after the final dose has been taken. In normal use, the indexer is driven via a cam member, which is in turn driven by a yoke, which is connected to the cap. Although the disc is prevented from being advanced after the final dose has been taken, it is not clear whether the cap, yoke, cam or breath flap can be moved normally as the cap is closed. It is unlikely that all would be blocked from moving since this would mean that the cap could not be closed; a user may be inclined to force the cap if he or she finds it is resisting being closed; this may result in the mechanism failing in some way.

### SUMMARY OF THE INVENTION

An object of the present invention is to reduce the risk of a user wrongly believing that a dose of medicament has been inhaled. This and other objects, which will become apparent in the following, are accomplished by the inhaler defined in the accompanied claims.

The present invention is based on the insight that, when a primed inhaler is fired, the movement of the components involved in the medicament dispensing action commonly generate a short audible sound and that the user may get used to hearing that sound when inhaling. Thus, by muting said sound after all the doses have been dispensed, even though the user may attempt to inhale again, he/she will not hear the accustomed sound, thereby increasing the probability that the user would realise that no dose was dispensed during the last inhalation attempt. This is reflected in a first aspect of the invention.

The present invention is also based on the insight that, a patient may become accustomed to view a certain indicia of a status indicator which confirms that an inhaler is primed. By removing or changing said indicia after all the doses have been dispensed, the user will be more likely to notice that there are no doses left to be dispensed. This is reflected in a second aspect of the invention.

According to the first aspect of the invention, an inhaler is provided. The inhaler comprises

an outlet, such as a mouthpiece or a nasal adapter,

a plurality of sealed compartments containing doses of medicament to be dispensed through said outlet,

a dispensing mechanism having a primed state, in which it is ready for dispensing a dose, and a fired state, in which it has dispensed a dose, wherein the dispensing mechanism moves from its primed state to its fired state in response to an inhalation flow, wherein the dispensing mechanism generates a sound when it moves from its primed state to its fired state, and

a disabler which, after the final dose has been dispensed, is activated to disable the dispensing mechanism from generating said sound.

The disabler may be configured to function in any one of various ways. For instance, the disabler may prevent the dispensing mechanism from firing, e.g. by locking it in its primed state and prevent it form moving to its fired state. Another alternative, rather than preventing firing, would be for the disabler to provide a sound damping effect, such as a cushion which moderates the motion of the dispensing mechanism. Yet another alternative, is represented by at least one example embodiment, wherein the disabler, when activated, is configured to prevent the dispensing mechanism from reaching the primed state. Thus, this last-mentioned alternative prevents firing by not even allowing the dispensing mechanism to reach the primed state.

According to at least one example embodiment, the dispensing mechanism comprises

an opening mechanism (opening device) having an energized position in which it is biased towards an unloaded position, wherein during movement from the energized position to the unloaded position the opening mechanism opens a sealed compartment aligned with the outlet, and

a latch having a first position, in which it latches the opening mechanism in the energized position, and a second position, in which it allows the opening mechanism to be in said unloaded position, wherein the latch is at least partly arranged in a flow path such that an inhalation flow through the flow path affects the latch to move from the first to the second position,

wherein when the disabler is activated it prevents the latch from reaching the first position. Thus, according to said embodiment, in the primed state of the dispensing mechanism, the opening mechanism is in the energized position and is latched by the latch, which is in its first position. When the disabler is activated it prevents the dispensing mechanism from reaching the primed state, because the latch cannot keep the opening mechanism in the energized position. In this context the expression “aligned with the outlet” should be understood as having provided the compartment in a position for inhalation of the contained medicament through the outlet. The outlet may be a mouthpiece or a nasal adaptor.

According to at least one example embodiment the latch comprises a pivotable element for changing between the first and second positions of the latch, wherein, when activated, the disabler is configured to pivot the latch away from its first position in order to prevent the opening mechanism from becoming latched.

According to at least one example embodiment, the latch is biased towards its first position. The extent of the bias is suitably balanced against the expected airflow inducible by a user's inhalation. Thus, when an airflow exceeds a certain threshold the biasing force is overcome and the latch is moved to its second position. When the airflow drops under the threshold, the latch may return to its biased first position, however, there may be provided mechanisms for temporarily preventing such return motion in order to allow other parts (e.g. the opening mechanism and the compartments) of the inhaler to move before latching takes place. Eventually, the latch will be allowed to move to the first position for latching the opening mechanism in its energized position.

Although a specifically designated user control may be provided for operating the inhaler, e.g. a separate lever or button at the inhaler housing, suitably, the movement of an outlet cover may be used for priming the inhaler. This is reflected in at least one example embodiment of the invention, according to which the inhaler comprises an outlet cover movable for alternatingly closing and opening the outlet, the outlet cover being operatively connectible to the dispensing mechanism in order to, upon one of said closing or opening movements of the outlet cover, move the dispensing mechanism to the primed state, wherein, when the disabler is activated, the dispensing mechanism is prevented from reaching the primed state despite said movements of the outlet cover.

Rather than affecting the latch, another alternative for the disabler would be to prevent the actual opening mechanism from reaching its energized position, which would likewise result in the dispensing mechanism not becoming primed. Yet another alternative for preventing the priming of the dispensing mechanism, would be to prevent the functioning of any means used for energizing the opening mechanism, e.g. if the closing of an outlet cover would normally result in the moving the opening mechanism to the opening position, the disabler could prevent the outlet cover from closing or could disable the connection between the outlet cover and the opening mechanism.

The disabler may be activated in various manners. For instance, it may be activated electronically or mechanically. According to at least one example embodiment the inhaler comprises a base having said plurality of sealed compartments and being provided with an activator. An indexing mechanism (indexing device) moves the base in order to sequentially align the compartments with the outlet. When the indexing mechanism indexes the base after the final dose has been dispensed, the activator activates the disabler to disable the dispensing mechanism from generating said sound.

For such a base, each compartment may represent a dose count position of the base, wherein the base, in addition to said dose count positions comprises a blank position which the indexing mechanism aligns with the outlet after all the dose count positions have been aligned with the outlet. When the indexing mechanism aligns the blank position with the outlet the activator activates the disabler.

In the case of the base being in the form of a rotatable disk, which will later be discussed in more detail, the activator may suitably be in the form of a protrusion, such as a peg, which is rotatable with the base. By enabling the activator to rotate with the disk, it may be moved one rotational step for each indexing of the base. After all the doses have been taken, the next rotational movement of the base causes the protrusion to interact with the disabler. For instance, the activator could be a protrusion on the outer enveloping surface of the base, which eventually will push the disabler to serve its purpose. Alternatively, the activator could be an indentation in the enveloping surface and the disabler could have a matching portion which is biased towards the enveloping surface and snaps into the indentation when they are moved in register with each other. Other alternatives are also conceivable. For instance, the activator may be located elsewhere on the base or not even be provided on the base. Furthermore, the corresponding functions could also be implemented for other types of bases than rotatable disks. Thus, in general terms without being limited to a specific base configuration, according to at least one example embodiment, the disabler has a disabling position, in which it prevents the dispensing mechanism from generating said sound, and a removed position, in which it allows the dispensing mechanism to generate said sound, wherein the activator is mounted on the base and adapted to move (e.g. push) the disabler to its disabling position.

As should be understood from the discussion above, although the inventive idea may be implemented for various base configurations, be it in the form of a strip or otherwise shaped supporting structure, the rotatable disk configuration is envisaged in accordance with at least one example embodiment. Thus, the base may comprise a rotatable disk provided with a circumferentially-oriented sequence of cavities, each cavity being sealed by a respective foil portion.

According to at least one example embodiment each foil portion is attached to a respective separating element for separating the foil portion from the cavity, wherein upon rotation of the disk the separating element next in turn is presented to the opening mechanism for removal of the separating element and the attached foil portion.

The opening mechanism may comprise an actuator which is engagable with the separating element to cause the separating element to be moved away from the cavity. Upon rotation of the disk the separating element next in turn is presented to the actuator. The rotatable disk may be connected to a separate manually operable lever. An alternative is to connect the rotation of the disk to the movement of the outlet cover. Thus, in either the course of opening or closing the outlet cover, the disk is rotated, thereby indexing the inhaler one step to the next dose. For instance, in an embodiment wherein the closing of the outlet cover causes the actuator to move to its energized position, the rotatable disk may also be moved (indexed) as a result of said closing.

In a multi-dose inhaler, the foil portions may be provided as one foil and, optionally, the foil portions may be defined by perforations or other material weakenings for facilitating removal of a foil portion from the cavity when the associated separating element is moved away from the base. As an alternative to a single foil, the foil portions may be applied in the form of individual patches. The foil portions may be attached to the base and the separating elements by welding, gluing or other suitable method. It should be noted that the terms “foil” and “foil portion” are not limited to a single material layer. On the contrary a foil or foil portion may comprise a plurality of layers. For instance, foil may comprise a metal layer which is coated with lacquer or polymer layer on one or both sides in any suitable combination in order to provide the desired stiffness, attachment capability, etc.

In order to separate a foil portion from the cavity it is sealing, the foil portion should be appropriately attached to its associated separating element. According to at least one example embodiment of the invention, the attachment force between the separating element and the respective associated foil portion is larger than the attachment force between the base and the foil portion, whereby movement of such a separating element away from its associated cavity causes the associated foil portion to become separated from the base.

Suitably, the contact area between a foil portion and its associated attached separating element is dimensioned in such way that no ruptured flow-obstructing foil parts will remain after the separation has occurred. In other words, the flow path downstream and upstream of the cavity opening should be free from any obstructing fringes of foil. Suitably, on the base, the flow path upstream and downstream of the cavity opening is completely foil free after the separation has occurred. This may be accomplished by designing the separating element with longer (or equal) extension in the flow path direction than that of the foil portion. Since the foil portion extends across the cavity opening in order to seal the cavity, the attached separating element should also extend at least across the cavity opening. As mentioned previously, the foil portions may form part of one covering foil provided with perforations or weakenings which define the foil portions. Such perforations would be present between the cavity openings, and when the foil portions are ruptured at those perforations or weakenings any fringes would be located laterally of the cavity viewed from a flow direction perspective, and consequently no obstructing fringes would be present upstream or downstream of the cavity.

There are various ways to obtain a larger attachment force at the separating element/foil portion interface than at the foil portion/base interface. According to at least one example embodiment of the invention, the contact surface between a separating element and its associated foil portion is larger than the contact surface between that foil portion and the base. In other words the separating element/foil portion interface is larger than the foil portion/base interface. If the separating element covers the entire foil portion, then the contact surface will automatically be larger between the separating element and the foil portion than the contact surface between the foil portion and the base, because the piece of the foil portion located directly above the cavity opening is not attached to anything and only the surrounding area of the foil is attached to the base.

Another way to obtain different attachment forces is considered in at least one other example embodiment of the invention. The foil portions may comprise a first coating layer to which the base is attached and a second coating layer to which the separating elements are attached, wherein the tensile strength of the second coating layer is larger than the tensile strength of the first coating layer. The layers can provide different bonding properties, e.g. welds of different types of material, or glues of different types or amounts, or any combination thereof.

Other ways to obtain the difference in attachment forces could be to provide the separating element with specially designed geometric features, e.g. grooves into which the foil may be attached or other features that e.g. pierce the foil to create a firm grip.

Although the foil portion may be folded into grooves of the separating element or otherwise curved around the separating element e.g. to increase the attachment area, the foil portion may suitably just be flat, i.e. only extending in a single plane parallel to the base. This enables a simple assembling of the separating elements to the foil portions. When they have become assembled the foil may be attached to the base. An alternative would be to first attach the foil portions to the base, and then attach the separating elements onto the respective foil portions.

Suitably, the stiffness of the separating elements is substantially larger than the stiffness of the foil portions, wherein the separating elements enable the foil portions to perform a rigid body motion, and may thus become lifted or snapped off the base rather than peeled off.

Although the above exemplified embodiments have discussed one cavity having one associated separating element, an alternative would be to have two cavities having one common associated separating element. For instance, if two incompatible drug components are to be inhaled essentially simultaneously, they may suitably be provided in two separate cavities. The two cavities may be covered and sealed by one common foil portion (or one foil portion each), which in turn is attached to a common associated separating element extending across both cavities. Thus, when the separating element is moved away from the cavity, it will bring along the foil portion, uncovering both cavities from which the drug components can be entrained in an inhalation flow. The cavities could either be located in series in the base, i.e. one cavity being downstream of the other one, or they could be located in parallel, i.e. the inhalation flow reaches the cavities essentially simultaneously.

Although embodiments comprising cavities covered by foil portions and attached separating elements have been described in detail, the inventive idea may, of course, be used with other types of dry powder inhalers in which premetered doses of medicament are provided. Likewise, the dispensing mechanisms described in this application are merely illustrative examples. Thus, it should be understood that a disabler may be provided in connection with various variants of dispensing mechanisms and compartment arrangements.

According to at least one example embodiment, the inhaler comprises a status indicator for indicating to the user whether the inhaler is ready or not ready for inhalation, wherein when the disabler is activated, the status indicator is prevented from maintaining a ready indication.

According to at least one example embodiment, the status indicator is connected to the indexing mechanism to follow its motions, wherein when the indexing mechanism indexes the base the status indicator changes its indication from not ready to ready, wherein when the disabler disables the dispensing mechanism, the status indicator is prevented from maintaining the ready indication. In other words, when the disabler disables the dispensing mechanim the status indicator will, even if it is temporarily changed to “ready”, return to its “not ready” indication, e.g. in connection with the opening of the outlet cover, as will be exemplified with regard to the detailed description of the accompanying drawings.

According to at least one example embodiment, the indexing means is adapted to index the base after the opening mechanism has been moved from the unloaded position to the energized position. When the disabler prevents the latch from latching the opening mechanism in the energized position the opening mechanism is enabled to return to the unloaded position. In said at least one example embodiment, the opening mechanism is connected to the indexing mechanism in such manner that when the opening mechanism moves from the energized position to the unloaded position the indexing mechanism is set in reverse motion and thereby the status indicator changes from ready to not ready.

According to the second aspect of the invention, an inhaler is provided, the inhaler comprising

an outlet, such as a mouthpiece or a nasal adapter,

a display for presenting a status indicator to a user, the status indicator having a first indicia representing a ready to inhale status of the inhaler and a second indicia representing a not ready to inhale status of the inhaler,

an outlet cover movable between a first position in which it covers the outlet and a second position in which the outlet is uncovered, wherein, when said outlet cover is in its first position the status indicator is out of view to the user, and wherein, when said outlet cover is in its second position the status indicator is visible through said display,

a dispensing mechanism having a primed state, in which it is ready for dispensing a dose, and a fired state, in which it has dispensed a dose, wherein, when the dispensing mechanism is in its primed state the first indicia of the status indicator is aligned with the display and when the dispensing mechanism is in its fired state the second indicia of the status indicator is aligned with the display, and

a disabler which, after the final dose has been dispensed, is activated to disable the dispensing mechanism from being in said primed state when the outlet cover is in its second position, the display thereby presenting the second indicia of the status indicator to the user.

It should be understood that the inhaler according to the second aspect of the invention encompasses any embodiments or any features described in connection with the inhaler according to the first aspect of the invention, as long as those embodiments or features are compatible with the inhaler of the second aspect.

The medicament in the inhaler may comprise various active ingredients. The active ingredient may be selected from any therapeutic or diagnostic agent. For example, the active ingredient may be an antiallergic, a bronchodilator (e.g. a beta2-adrenoceptor agonist or a muscarinic antagonist), a bronchoconstrictor, a pulmonary lung surfactant, an analgesic, an antibiotic, a mast cell inhibitor, an antihistamine, an anti-inflammatory, an antineoplastic, an anaesthetic, an anti-tubercular, an imaging agent, a cardiovascular agent, an enzyme, a steroid, genetic material, a viral vector, an antisense agent, a protein, a peptide, a non-steroidal glucocorticoid Receptor (GR Receptor) agonist, an antioxidant, a chemokine antagonist (e.g. a CCR1 antagonist), a corticosteroid, a CRTh2 antagonist, a DP1 antagonist, an Histone Deacetylase Inducer, an IKK2 inhibitor, a COX inhibitor, a lipoxygenase inhibitor, a leukotriene receptor antagonist, an MPO inhibitor, a p38 inhibitor, a PDE inhibitor, a PPARγ agonist, a protease inhibitor, a statin, a thromboxane antagonist, a vasodilator, an ENAC blocker (Epithelial Sodium-channel blocker) and combinations thereof

Examples of specific active ingredients that can be incorporated in the inhaler include:

(i) antioxidants:- Allopurinol, Erdosteine, Mannitol, N-acetyl cysteine choline ester, N-acetyl cysteine ethyl ester, N-Acetylcysteine, N-Acetylcysteine amide and Niacin;
(ii) chemokine antagonists:- BX471 ((2R)-1-[[2-[(aminocarbonyl)amino]-4-chlorophenoxy]acetyl]-4-[(4-fluorophenyl)methyl]-2-methylpiperazine monohydrochloride), CCX634, N-{2-[((2S)-3-{[1-(4-chlorobenzyl)piperidin-4-yl]amino}-2-hydroxy-2-methylpropyl)oxy]-4-hydroxyphenyl} acetamide (see WO 2003/051839), and 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid (see WO2008/010765), 656933 (N-(2-bromophenyl)-N′-(4-cyano-1H-1,2,3-benzotriazol-7-yl)urea), 766994 (4-({[({[(2R)-4-(3,4-dichlorobenzyl)morpholin-2-yl]methyl} amino) carbonyl]-amino}methyl)benzamide), CCX-282, CCX-915, Cyanovirin N, E-921, INCB-003284, INCB-9471, Maraviroc, MLN-3701, MLN-3897, T-487 (N-{1-[3-(4-ethoxyphenyl)-4-oxo-3 ,4-dihydropyrido [2,3 -d]pyrimidin-2-yl] ethyl}-N-(pyridin-3-ylmethyl)-2-[4-(trifluoromethoxy)phenyl]acetamide) and Vicriviroc
(iii) Corticosteroids: -Alclometasone dipropionate, Amelometasone, Beclomethasone dipropionate, Budesonide, Butixocort propionate, Ciclesonide, Clobetasol propionate, Desisobutyrylciclesonide, Etiprednol dicloacetate, Fluocinolone acetonide, Fluticasone Furoate, Fluticasone propionate, Loteprednol etabonate (topical) and Mometasone furoate.
(iv) DP1 antagonisits:- L888839 and MK0525;
(v) Histone deacetylase inducers:- ADC4022, Aminophylline, a Methylxanthine or Theophylline;
(vi) IKK2 inhibitors:- 2-{[2-(2-Methylamino-pyrimidin-4-yl)-1H-indole-5-carbonyl]-amino}-3-(phenyl-pyridin-2-yl-amino)-propionic acid;
(vii) COX inhibitors:- Celecoxib, Diclofenac sodium, Etodolac, Ibuprofen, Indomethacin, Meloxicam, Nimesulide, OC1768, OC2125, OC2184, OC499,OCD9101, Parecoxib sodium, Piceatannol, Piroxicam, Rofecoxib and Valdecoxib;
(viii) Lipoxygenase inhibitors:- Ajulemic acid, Darbufelone, Darbufelone mesilate,

Dexibuprofen lysine (monohydrate), Etalocib sodium, Licofelone, Linazolast, Lonapalene, Masoprocol, MN-001 , Tepoxalin, UCB-35440, Veliflapon, ZD-2138, ZD-4007 and Zileuton ((±)-1-(1-Benzo[b]thien-2-ylethyl) -1-hydroxyurea);

(ix) Leukotriene receptor antagonists:- Ablukast, Iralukast (CGP 45715A), Montelukast, Montelukast sodium, Ontazolast, Pranlukast, Pranlukast hydrate (mono Na salt), Verlukast (MK-679) and Zafirlukast;
(x) MPO Inhibitors:- Hydroxamic acid derivative (N-(4-chloro-2-methyl -phenyl)-4-phenyl-4-[[(4-propan-2-ylphenyl)sulfonylamino]methyl]piperidine -1-carboxamide), Piceatannol and Resveratrol;
(xi) Beta2-adrenoceptor agonists:- metaproterenol, isoproterenol, isoprenaline, is albuterol, salbutamol (e.g. as sulphate), formoterol (e.g. as fumarate), salmeterol (e.g. as xinafoate), terbutaline, orciprenaline, bitolterol (e.g. as mesylate), pirbuterol, indacaterol, salmeterol (e.g. as xinafoate), bambuterol (e.g. as hydrochloride), carmoterol, indacaterol (CAS no 312753-06-3; QAB-149), formanilide derivatives e.g. 3-(4-{[6-({(2R)-2-[3-(formylamino)-4-hydroxyphenyl]-2-hydroxyethyl} amino)hexyl]oxy}-butyl)-benzenesulfonamide; 3-(4-{[6-({(2R)-2-hydroxy-2-[4-hydroxy-3-(hydroxy-methyl)phenyl]ethyl} amino)-hexyl]oxy}butyl)benzenesulfonamide; GSK 159797, GSK 159802, GSK 597901, GSK 642444, GSK 678007; and a compound selected from N-[2-(Diethylamino)ethyl]-N-(2-{[2-(4-hydroxy -2-oxo-2,3 -dihydro-1,3 -benzothiazol-7-yl)ethyl]amino}ethyl)-3-[2-(1-naphthyl)ethoxy]propanamide, N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chloropheny)ethoxy]propanamide, 7-[(1R)-2-({2-[(3-{[2-(2-Chloropheny)ethyl]amino}propyl)thio]ethyl}amino)-1-hydroxyethyl]-4-hydroxy-1,3-benzothiazol-2(3H)-one, and N-Cyclohexyl-N3-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. wherein the counter ion is hydrochloride (for example a monohydrochloride or a dihydrochloride), hydrobromide (for example a monohydrobromide or a dihydrobromide), fumarate, methanesulphonate, ethanesulphonate, benzenesulphonate, 2,5-dichlorobenzenesulphonate, ρ-toluenesulphonate, napadisylate (naphthalene-1,5-disulfonate or naphthalene -1-(sulfonic acid)-5-sulfonate), edisylate (ethane-1,2-disulfonate or ethane -1-(sulfonic acid)-2-sulfonate), D-mandelate, L-mandelate, cinnamate or benzoate.)
(xii) Muscarinic antagonists:- Aclidinium bromide, Glycopyrrolate (such as R,R-, R,S-, S,R-, or S,S-glycopyrronium bromide), Oxitropium bromide, Pirenzepine, telenzepine, Tiotropium bromide, 3(R)-1-phenethyl-3-(9H-xanthene -9-carbonyloxy)-1-azoniabicyclo[2.2.2]octane bromide, (3R)-3-[(2S)-2-cyclopentyl-2-hydroxy-2-thien-2-ylacetoxy]-1-(2-phenoxyethyl)-1-azoniabicyclo[2.2.2]actane bromide, a quaternary salt (such as [2-((R)-Cyclohexyl-hydroxy-phenyl-methyl)-oxazol-5-ylmethyl]-dimethyl-(3-phenoxy-propyl)-ammonium salt, [2-(4-Chloro-benzyloxy)-ethyl]-[2-((R)-cyclohexyl-hydroxy-phenyl-methyl)-oxazol-5-ylmethyl]- dimethyl-ammonium salt and (R)-1-[2-(4-Fluoro-phenyl)-ethyl]-3-((S)-2-phenyl-2-piperidin-1-yl-propionyloxy)-1-azonia-bicyclo[2.2.2]octane salt wherein the counter-ion is, for example, chloride, bromide, sulfate, methanesulfonate, benzenesulfonate (besylate), toluenesulfonate (tosylate), napthalenebissulfonate (napadisylate or hemi-napadisylate), phosphate, acetate, citrate, lactate, tartrate, mesylate, maleate, fumarate or succinate)
(xiii) p38 Inhibitors:- 681323, 856553, AMG548 (2-[[(2S)-2-amino-3-phenylpropyl]amino]-3-methyl-5-(2-naphthalenyl)-6-(4-pyridinyl)-4(3H)-pyrimidinone), Array-797, AZD6703, Doramapimod, KC-706, PH 797804, R1503, SC-80036, SCIO469, 6-chloro-5-[[(2S,5R)-4-[(4-fluorophenyl)methyl]-2,5-domethyl-1-piperazinyl]carbonyl]-N,N,1-trimethyl-α-oxo-1H-indole-3-acetamide, VX702 and VX745 (5-(2,6-dichlorophenyl)-2-(phenylthio)-6H-pyrimido[1,6-b]pyridazin-6-one);
(xiv) PDE Inhibitors:- 256066, Arofylline (3-(4-chlorophenyl)-3,7-dihydro-1-propyl-1H-Purine-2,6-dione), AWD 12-281 (N-(3,5-dichloro-4-pyridinyl)-1-[(4-fluorophenyl)methyl]-5-hydroxy-α-oxo-1H-indole-3-acetamide), BAY19-8004 (Bayer), CDC-801 (Calgene), Celgene compound ((βR)-β-(3,4-dimethoxyphenyl)-1,3-dihydro-1-oxo-2H-isoindole-2-propanamide), Cilomilast (cis-4-cyano-4- [3 -(cyclopentyloxy)-4-methoxyphenyl]-cyclohexanecarboxylic acid), 2-(3,5-dichloro-4-pyridinyl)-1-(7-methoxyspiro[1,3-benzodioxole -2,1′-cyclopentan]-4-yl)ethanone (CAS number 185406-34-2)), (243,4-difluorophenoxy)-5-fluoro-N-[cis-4-[(2-hydroxy-5-methylbenzoyl)amino]cyclohexyl]-)-3-pyridinecarboxamide), (2-(3,4-difluorophenoxy)-5-fluoro-N-[cis-4-[[2-hydroxy-5-(hydroxymethyl)benzoyl]amino]cyclohexyl]-3-pyridinecarboxamide,), CT2820, GPD-1116, Ibudilast, IC 485, KF 31334, KW-4490, Lirimilast ([242,4-dichlorobenzoyl)-6-[(methylsulfonyl)oxy]-3-benzofuranyl])-urea), (N-cyclopropyl-1,4-dihydro-4-oxo-1-[3-(3-pyridinylethynyl)phenyl]-)-1,8-naphthyridine-3-carboxamide), (N-(3,5-dichloro-4-pyridinyl) -4-(difluoromethoxy)-8-[(methylsulfonyl)amino])-1-dibenzofurancarboxamide), ONO6126, ORG 20241 (4-(3,4-dimethoxyphenyl)-N-hydroxy-) -2-thiazolecarboximidamide), PD189659/PD168787 (Parke-Davis), Pentoxifylline (3,7-dihydro-3,7-dimethyl-1-(5-oxohexyl)-)-1H-purine-2,6-dione), compound (5-fluoro-N-[4-[(2-hydroxy-4-methyl-benzoyl)amino]cyclohexyl]-2-(thian -4-yloxy)pyridine-3-carboxamide), Piclamilast (3-(cyclopentyloxy)-N -(3,5-dichloro-4-pyridinyl)-4-methoxy-benzamide), PLX-369 (WO 2006026754), Roflumilast (3-(cyclopropylmethoxy)-N-(3,5-dichloro-4-pyridinyl) -4-(difluoromethoxy)benzamide), SCH 351591 (N-(3,5-dichloro-l-oxido -4-pyridinyl)-8-methoxy-2-(trifluoromethyl)-5-quinolinecarboxamide), Se1CID(TM) CC-10004 (Calgene), T-440 (Tanabe), Tetomilast (6-[2-(3,4-diethoxyphenyl)-4-thiazolyl]-2-pyridinecarboxylic acid), Tofimilast (9-cyclopentyl -7-ethyl-6,9-dihydro-3-(2-thienyl)-5H-pyrazolo[3,4-c]-1,2,4-triazolo[4,3-a]pyridine), TPI 1100, UCB 101333-3 (N,2-dicyclopropyl-6-(hexahydro-1H-azepin-l-yl)-5-methyl-4-pyrimidinamine), V-11294A (Napp), VM554/VM565 (Vernalis), and Zardaverine (6-[4-(difluoromethoxy)-3-methoxyphenyl]-3(2H)-pyridazinone).
(xv) PDE5 Inhibitors:- Gamma-glutamyl[s-(2-iodobenzyl)cysteinyl]glycine, Tadalafil, Vardenafil, sildenafil, 4-phenyl-methylamino-6-chloro-2-(1-imidazolyl)-quinazoline, 4-phenyl-methylamino-6-chloro-2-(3-pyridyl)-quinazoline, 1,3-dimethyl-6-(2-propoxy-5-methanesulphonylamidophenyl) -1,5-dihydropyrazolo[3,4-d]pyrimidin-4-one and 1-cyclopentyl-3-ethyl-6-(3-ethoxy-4-pyridyl)-pyrazolo[3,4-d]pyrimidin-4-one;
(xvi) PPARγ agonists:- Pioglitazone, Pioglitazone hydrochloride, Rosiglitazone Maleate, Rosiglitazone Maleate ((-)-enantiomer, free base), Rosiglitazone maleate/Metformin hydrochloride and Tesaglitizar;
(xvii) Protease Inhibitors:- Alpha1-antitrypsin proteinase Inhibitor, EPI-HNE4, UT-77, ZD-0892, DPC-333, Sch-709156 and Doxycycline;
(xviii) Statins:- Atorvastatin, Lovastatin, Pravastatin, Rosuvastatin and Simvastatin
(xix) Thromboxane Antagonists: Ramatroban and Seratrodast;
(xx) Vasodilators:- A-306552, Ambrisentan, Avosentan, BMS-248360, BMS-346567, BMS-465149, BMS-509701, Bosentan, BSF-302146 (Ambrisentan), Calcitonin Gene-related Peptide, Daglutril, Darusentan, Fandosentan potassium, Fasudil, Iloprost, KC-12615 (Daglutril), KC-12792 2AB (Daglutril), Liposomal treprostinil, PS-433540, Sitaxsentan sodium, Sodium Ferulate, TBC-11241 (Sitaxsentan), TBC-3214 (N-(2-acetyl-4,6-dimethylphenyl)-3-[[(4-chloro-3-methyl-5-isoxazolyl)amino]sulfonyl]-2-thiophenecarboxamide), TBC-3711, Trapidil, Treprostinil diethanolamine and Treprostinil sodium;
(xxi) ENACs:- Amiloride, Benzamil, Triamterene, 552-02, PSA14984, PSA25569, PSA23682 and AER002.

The inhaler may contain a combination of two or more active ingredients, for example a combination of two or more of the specific active ingredients listed in (i) to (xxi) herein above.

In one embodiment the inhaler contains an active ingredient selected from mometasone, ipratropium bromide, tiotropium and salts thereof, salemeterol, fluticasone propionate, beclomethasone dipropionate, reproterol, clenbuterol, rofleponide and salts, nedocromil, sodium cromoglycate, flunisolide, budesonide, formoterol fumarate dihydrate, terbutaline, terbutaline sulphate, salbutamol base and sulphate, fenoterol, 3-[2-(4-Hydroxy-2-oxo-3H-1,3-benzothiazol-7-yl)ethylamino]-N-[2-[2-(4-methylphenyl)ethoxy]ethyl]propane-sulphonamide, hydrochloride, indacaterol, aclidinium bromide, N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide); N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. di-D-mandelate); a [2-(4-Chloro-benzyloxy)-ethyl]-[2-((R)-cyclohexyl-hydroxy -phenyl-methyl)-oxazol-5-ylmethyl]- dimethyl-ammonium salt (e.g. hemi-naphthalene -1,5-disulfonate); a (R)-1-[2-(4-Fluoro-phenyl)-ethyl]-3-((S)-2-phenyl-2-piperidin-1-yl-propionyloxy)-1-azonia-bicyclo[2.2.2]octane salt (e.g. bromide or toluenesulfonate); or a combination of any two or more thereof.

Specific combinations of active ingredients which may be incorporated in the inhaler include:—

(a) formoterol (e.g. as fumarate) and budesonide;

(b) formoterol (e.g. as fumarate) and fluticasone;

(c) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide) and a [2-(4-Chloro-benzyloxy)-ethyl]-[2-((R)-cyclohexyl-hydroxy-phenyl-methyl)-oxazol-5-ylmethyl]-dimethyl-ammonium salt (e.g. hemi-naphthalene-1,5-disulfonate);

(d) N-[2-(Diethylamino)ethyl]-N-(2-{[2-(4-hydroxy-2-oxo-2,3 -dihydro-1,3-benzothiazol-7-yl)ethyl]amino}ethyl)-3-[2-(1-naphthyl)ethoxy]propanamide or a pharmaceutically acceptable salt thereof (e.g. dihydrobromide) and a (R)-1-[2-(4-Fluoro-phenyl)-ethyl]-3-((S)-2-phenyl-2-piperidin-1-yl-propionyloxy) -1-azonia-bicyclo[2.2.2]octane salt (e.g. bromide or toluenesulfonate);

(e) N-Cyclohexyl-N³-[2-(3-fluorophenyl)ethyl]-N-(2-{[2-(4-hydroxy-2-oxo -2,3-dihydro-1,3-benzothiazol-7-yl)ethyl]amino}ethyl)-β-alaninamide or a pharmaceutically acceptable salt thereof (e.g. di-D-mandelate) and [2 -(4-Chloro-benzyloxy)-ethyl]-[2-((R)-cyclohexyl-hydroxy-phenyl-methyl)-oxazol -5-ylmethyl]-dimethyl-ammonium salt (e.g. hemi-naphthalene-1,5-disulfonate);

(f) N-Cyclohexyl-N³[2-(3-fluorophenyl)ethyl]-N-(2-{[2-(4-hydroxy-2 -oxo-2,3-dihydro-1,3-benzothiazol-7-yl)ethyl]amino}ethyl)-β-alaninamide or a pharmaceutically acceptable salt thereof (e.g. di-D-mandelate) and a (R)-1-[2-(4-Fluoro-phenyl)-ethyl]-3-((S)-2-phenyl-2-piperidin-1-yl-propionyloxy) -1-azonia-bicyclo[2.2.2]octane salt (e.g. bromide or toluenesulfonate).

### BRIEF DESCRIPTION OF THE DRAWINGS

FIG. 1 is an exploded view of an inhaler according to at least one example embodiment of the invention.

FIG. 2 is a cross-sectional view of selected details of the inhaler.

FIG. 3 illustrates, at the time of dispensing medicament from the inhaler, a cross-sectional view of selected details of the inhaler.

FIGS. 4 to 8 and 11 illustrate various details of the inhaler.

FIG. 9 is a cross-sectional view of selected details of the inhaler before indexing.

FIG. 10 is a cross-sectional view of selected details of the inhaler after indexing.

FIGS. 12, 13a, 13b, 14a, 14b, 15a and 15b illustrate the functioning of a disabler provided in the inhaler.

FIGS. 16 to 18 illustrate the functioning of a disabler in an alternative embodiment.

### DETAILED DESCRIPTION OF THE DRAWINGS

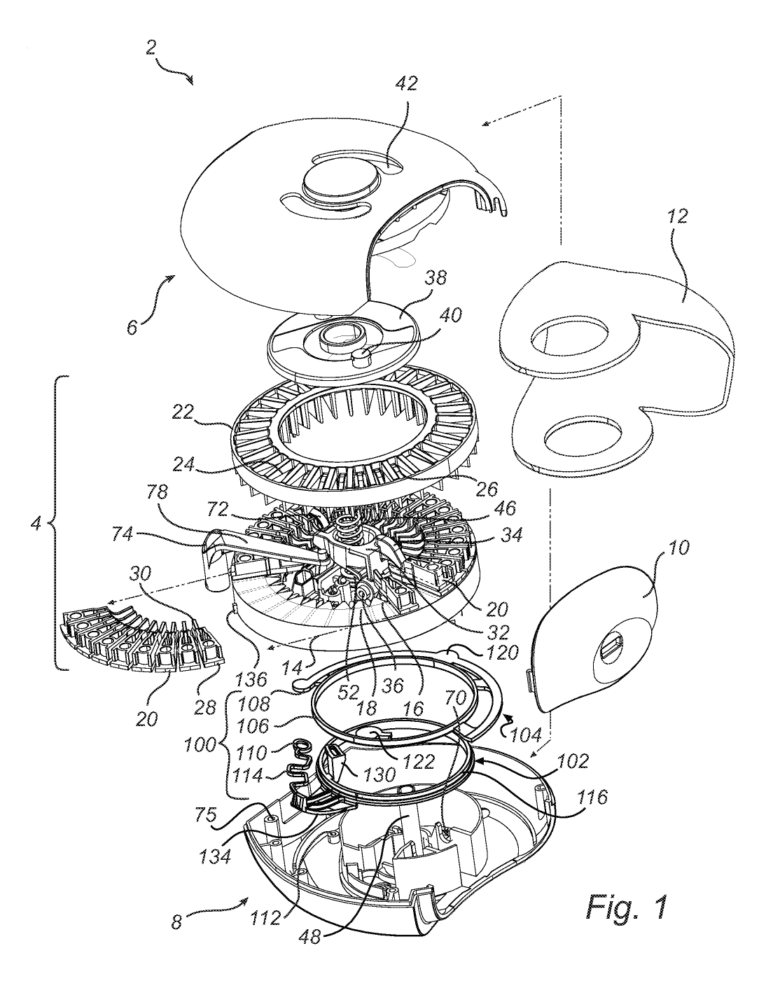

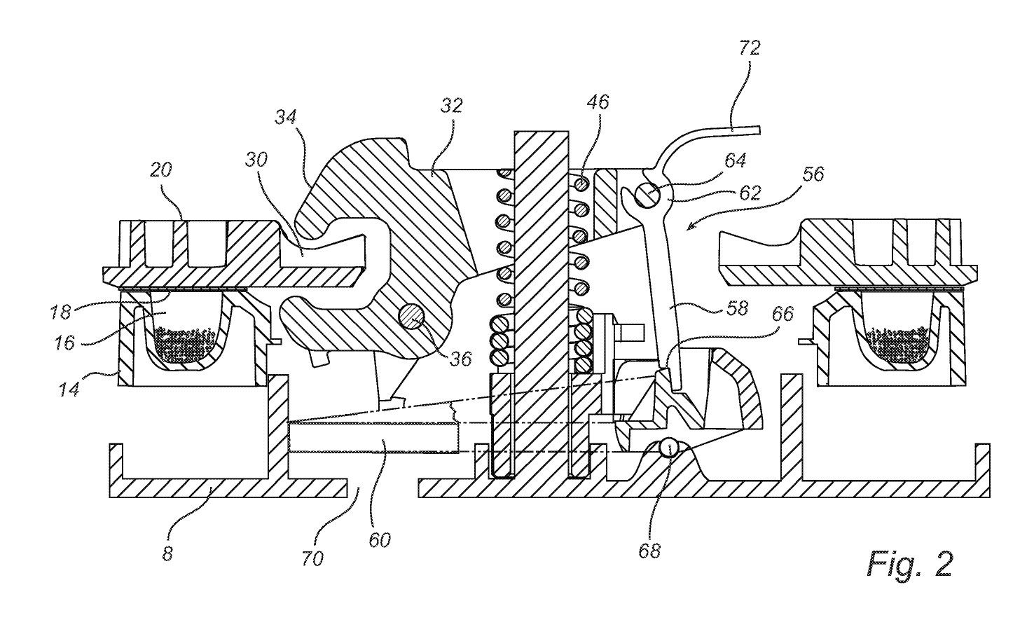

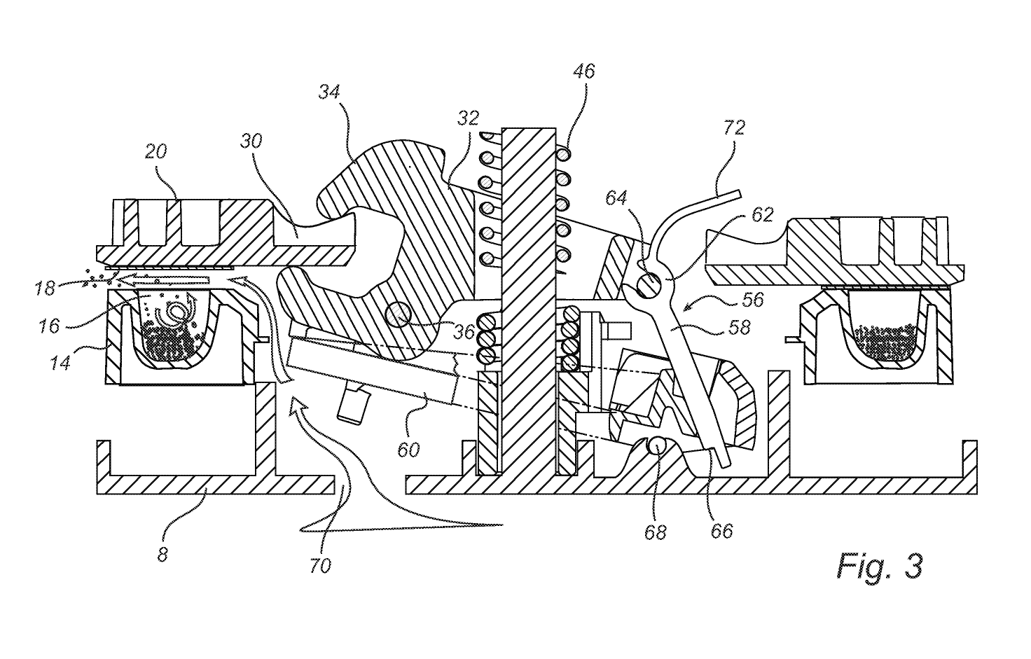

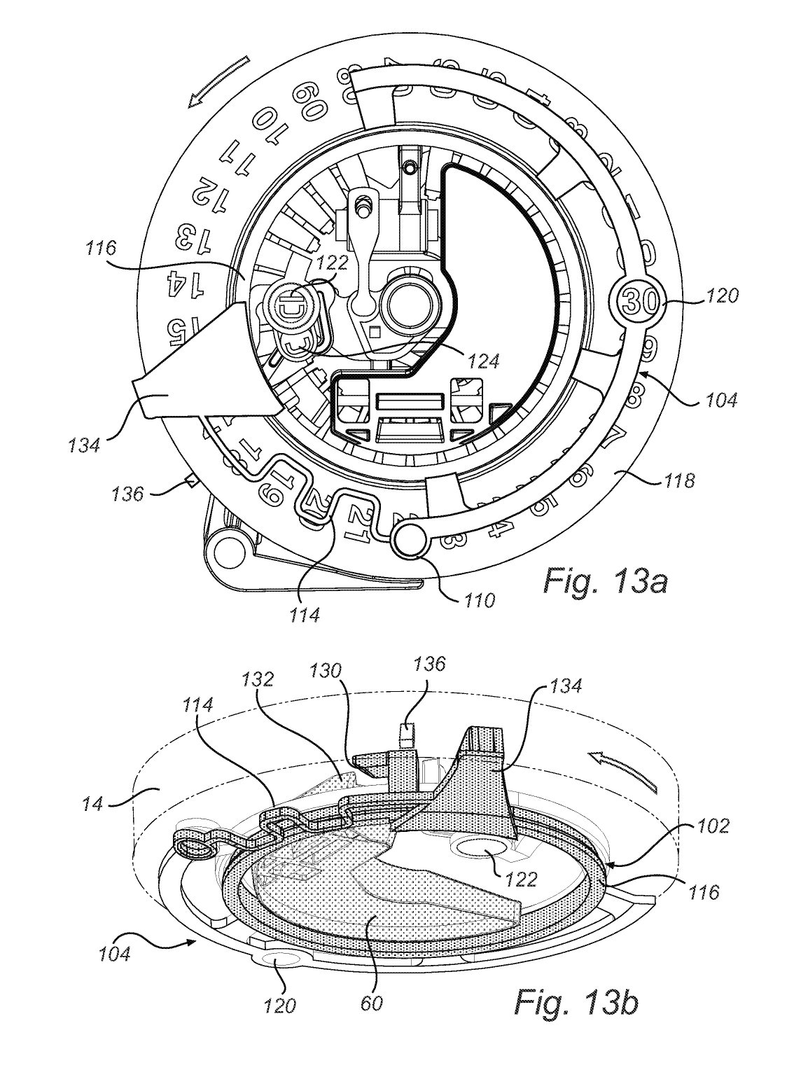

Before providing a detailed description of the various parts of the illustrated inhaler, there will first be provided a brief introduction focusing on the inventive concept in relation to the illustrated example embodiment. Accordingly, an inhaler 2 comprises an outlet in the form of a mouthpiece 10 (see FIG. 1). A base 14 has a plurality of sealed compartments in the form of sealed cavities 16 which are arranged to be sequentially aligned with and dispensed through the mouthpiece 10. Above each cavity 16, a respective associated separating element 20 is attached to the upper side of a foil portion 18. A dispensing mechanism comprises an actuator 32 for lifting the separating elements 20 and a latch 56 is provided to keep the actuator 32 in an energized position, whereby the dispensing mechanism is in a primed state (see FIG. 2) and ready for dispensing a dose. The dispensing mechanism also has a fired state (see FIG. 3) in which it has dispensed a dose, wherein the dispensing mechanism moves from its primed state to its fired state in response to an inhalation flow (see arrows in FIG. 3). The dispensing mechanism generates a sound when it moves from its primed state to its fired state. For instance, when the separating element 20 is lifted by the actuator 32 and hits another component of the inhaler, the impact may generate a sound, or the spring-induced movement of the actuator 32 may in itself generate a sound. Although the mechanically generated audible sound serves the purpose of the inventive idea, an alternative would be to complement it with or replace it with an electronically generated sound. A disabler 102 which, after the final dose has been dispensed, is activated to disable the dispensing mechanism from generating said sound. The functioning of the disabler 102 is best illustrated in FIGS. 13a, 13b, 14a, 14b, 15a and 15b. An activator, herein illustrated in the form of a peg 136 on the enveloping surface of the base 14, is rotatable with the base 14. After the final dose has been taken, the next time the base 14 is indexed, the peg 136 will hit a radially projecting portion 134 of the disabler 102, which will come into contact with a flap 60 of the above-mentioned latch 56. The flap 60 will thereby be lifted and disabled from latching the actuator 32. Thus, the dispensing mechanism can no longer reach its primed state, and consequently the sound which is otherwise generated when the dispensing mechanism moves from its primed state to its fired state is now prevented from being generated. Also, when the disabler 102 is activated a status indicator 124 becomes prevented from showing a “ready” status to the user. In the following, a more detailed description of the inhaler will be provided.

FIG. 1 is an exploded view of an inhaler 2 according to at least one example embodiment of the invention. The inhaler 2 comprises a dose dispensing assembly 4 having a general disk configuration, an upper housing portion 6, a lower housing portion 8, an outlet herein represented in the form of a mouthpiece 10, an outlet cover 12, and a disabler assembly 100.

The dose dispensing assembly 4 comprises a circular base 14 which has a plurality of sequentially arranged cavities 16 along the circular extension of the base. The cavities 16 can be provided with medicament, such as in dry powder form, and are sealed by foil portions 18, thus providing sealed compartments. The foil portions 18 are either part of one common foil or provided as separate patches. In the shown example, perforations have been provided to define the foil portions 18 and to facilitate separation from the base 14. Above each cavity 16, a respective associated separating element 20 is attached to the upper side of the foil portion 18. The separating elements 20 are attached by any suitable type of bonding, welding, gluing, etc. to the respective foil portions 18. Upwards movement or lifting of a separating element 20 causes the attached foil portion 18 to become separated from the cavity 16.

A circular guide structure 22 is provided above the separating elements 20. The guide structure 22 comprises a plurality of guide sections 24 divided by vertically extending walls, each guide section 24 being associated with a respective separating element 20. When a separating element 20 is lifted from the cavities-holding base 14, the associated guide section 24 will guide the upwards movement of the separating element 20. Each guide section 24 is provided with a counteracting element, such as a blade spring 26. When the separating element 20 is lifted so that it hits the blade spring 26 or some other portion of the guide structure 22, a sound is generated. After a separating element 20 has been lifted and medicament in the opened cavity 16 has been entrained in the inhalation airflow and the separating element 20 has returned to the base 14, the blade spring 26 will keep the lifted separating element 20 in contact with the base 14 to cover the cavity 16. This will make it difficult for any remaining powder to exit the covered used cavity 16, thus reducing the risk of dose variation which could occur if such remaining powder would be entrained in a following inhalation. It also reduces the risk of remaining powder exiting the cavity 16 and jamming mechanical components in the inhaler or the risk of the separating element creating a rattling noise which would be undesirable for the user. The vertical walls dividing the circular guide structure 22 into guide sections 24 function as lateral flow path defining elements. Thus, an inhalation airflow is prevented from deviating sideways once it reaches the cavity area of the base 14 and will be led to the mouthpiece 10. An alternative would be to have shorter vertical walls, in which case neighbouring separating elements 20 could have the function of lateral flow path defining elements.

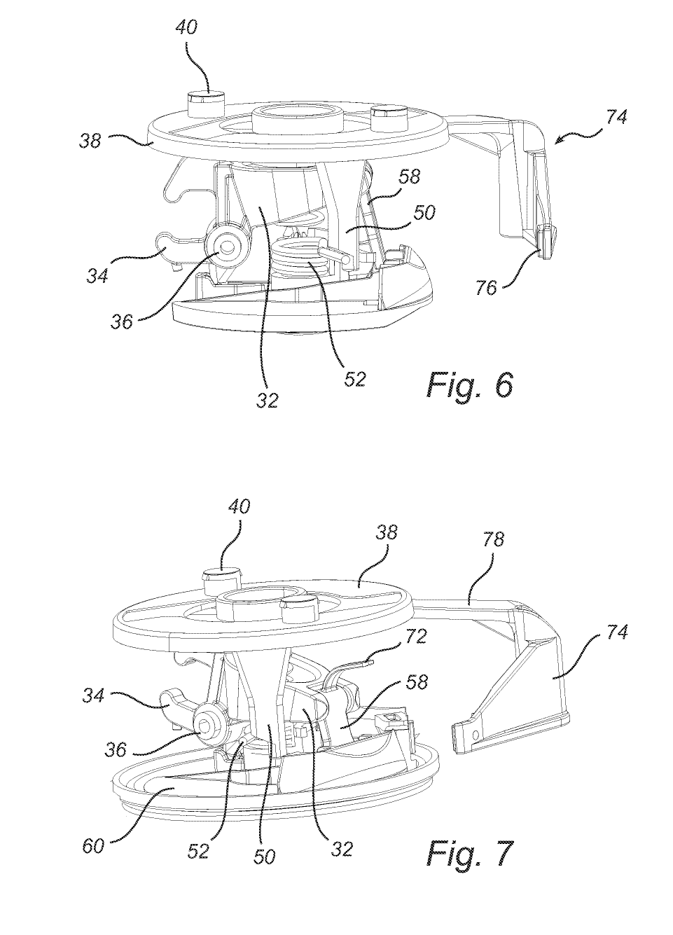

Each separating element 20 has a base-covering portion 28 which is in register with a respective cavity 16 in the base. Additionally, each separating element 20 has a centrally projecting portion 30. An opening mechanism comprising an actuator 32 for lifting the separating elements 20 is provided. The actuator is herein represented in the form of a pivotable lever provided with jaws 34 for gripping the centrally projecting portions 30 of the separating elements 20. The actuator 32 has an energized position (FIGS. 2 and 6) in which the jaws 34 are in a lowered position and, after pivoting about a pivot axel 36, an unloaded position (FIGS. 3 and 7) in which the jaws 34 are in a raised position. The actuator 32 with its jaws 34 is only pivotable around the horizontal axel 36 and will thus remain facing the mouthpiece 12 during operation of the inhaler 2.

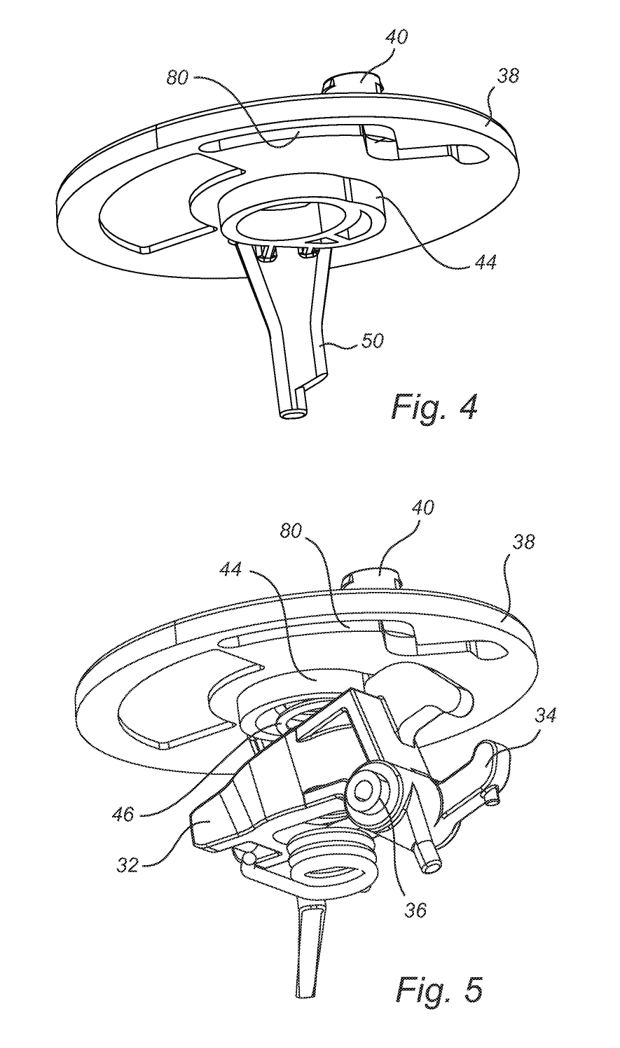

Returning to FIG. 1, a generally disk-shaped insert 38 is provided under the upper housing portion 6. The upper side of the insert 38 is provided with two pegs 40. The pegs 40 extend upwardly through respective arcuate openings 42 in the upper housing portion 6 and are connected to the outlet cover 12. As the outlet cover 12 is rotated, the pegs 40 will through the arcuate openings 42 transmit the motion to the insert 38 which will also rotate. The underside of the insert 38 is provided with a first force transmitting member, herein illustrated in the form of a cam 44 (see FIG. 4), which will convert the rotating motion to a linear force affecting the jaws 34 of the actuator 32 in order to return the actuator 32 from its unloaded position to its energized position. As the cam 44 comes into contact with the jaws 34 of the actuator 32 (see FIG. 5), the actuator 32 will be moved radially towards the separating element 20 and will rotate around its pivot axel 36. Also, the jaws 34 will drop down to the primed or energized position of the actuator 32 (see FIG. 2). The lowering of the jaws 34 will be against the force of a coil spring 46 which is biased to raise the jaws 34 to the unloaded position. The coil spring 46 is wound around a post 48 projecting upwardly from the lower housing portion 8.

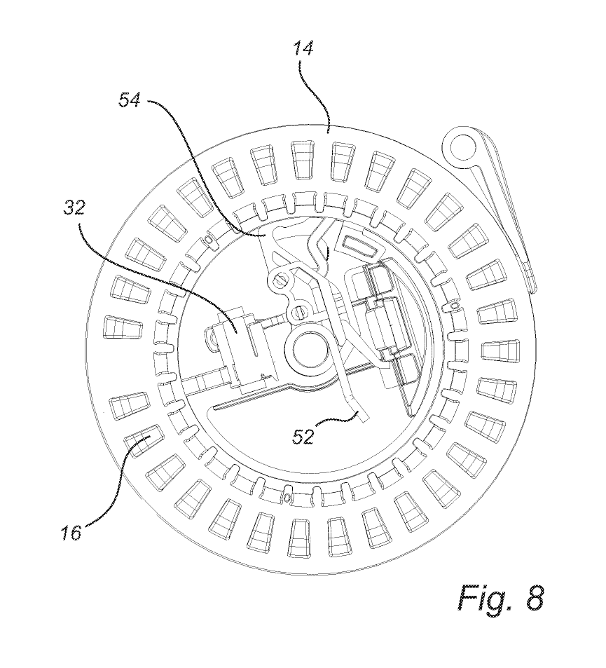

As illustrated in FIGS. 4, 6 and 7, the underside of the insert 38 is also provided with a projecting second force transmitting member 50 which is configured and adapted to engage an end of a torsion spring 52 located under the coil spring 46 and around the same post 48. The torsion spring 52 is connected to a drive member 54 for rotatingly advancing the cavities 16 by one increment at a time, so as to each time bring an unopened cavity in alignment with the mouthpiece 10. The drive member is best seen in FIGS. 8, 9, 10 and 11.

A latch 56 is provided to keep the actuator 32 in the energized position, which is clearer from FIG. 2. The latch 56 comprises a first element in the form of an elongated prop 58 and a second element in the form of a flap 60. The elongated prop 58 has a first end portion 62 which is pivotable around a first horizontal axle 64 near that end of the actuator 32 which is located distally to the mouthpiece 10 (the jaws 34 being located proximally to the mouthpiece 10). The elongated prop 58 has a second end portion 66 adapted to be supported by the flap 60. The flap 60 is pivotable around a second horizontal axle 68. The flap covers a number of air inlets 70 (FIGS. 1-3) provided in the lower housing portion 8. Air is allowed to enter the inhaler 2 through said air inlets 70 when the user inhales through the mouthpiece 10 (outlet).

FIG. 2 is a cross-sectional view of selected details of the inhaler, wherein the inhaler is in a primed state, i.e. the actuator 32 is latched in an energized position. Thus, the jaws 34 of the actuator 32 have been lowered against the force of the coil spring 46 and now enclose the centrally projecting portion 30 of a separating element 20 aligned with the mouthpiece. The second end portion 66 of the elongated prop 58 is supported by a mating portion of the flap 60. The latch 56 comprising the prop 58 and the flap 60 is now in its first position, in which it latches the actuator 32 in the energized position. The latch 56 is biased towards its first position. More specifically, in this exemplified embodiment, the interface or contact point between the second end portion 66 of the elongated prop 58 and the flap 60 is located on the same side of the second horizontal axle 68 as is the portion of the flap 60 covering the air inlets 70 (in FIG. 2, the contact point between the elongated prop 58 and the flap is located left of the second horizontal axle 68). Thus, the centre of mass and the force on the flap 60 provided by the elongated prop 58 will be located left (in FIG. 2) of the pivot point provided by the second horizontal axle 68, thereby keeping the flap 60 in the illustrated lowered position. As long as the flap 60 remains still, the prop 58 is also prevented from moving, thereby keeping the actuator 32 latched in its energized position. The force exerted on the flap 60 is suitably adjusted to correspond to an airflow threshold which is exceedable by a user's inhalation. A position-keeping element 72 is provided at the first end portion 62 of the prop 58. From above, the position-keeping element 72 will be in contact with the disk-shaped insert 38 (FIG. 1). That contact will ensure that the prop 58 does not accidentally pivot around the first horizontal axle 64 in case the user should turn the inhaler in a different orientation (e.g. upside down) when closing the outlet cover 12. Thus, the flap 60 and prop 58 will be able to latch the actuator 32 even if a user holds the inhaler upside down when closing the outlet cover 12.

In at least one other embodiment, the illustrated position-keeping element 72 could rather function as a biasing spring element 72. In such an embodiment, the biasing spring element 72, would not just be in contact with the disk-shaped insert 38 (FIG. 1), but would actually be pressed downwardly by the disk-shaped insert 38. This force exerted on the biasing spring element 72 would have a levering effect about the first axle 64, urging the second end portion 66 of the prop 58 in a direction towards the jaws 34 and the mouthpiece (clockwise rotation in FIG. 2). This urging of the second end portion 66, which is in contact with a mating portion of the flap 60, would keep the flap 60 biased in the illustrated substantially horizontal lowered position. The biasing force transmitted from the biasing spring element 72 to the flap 60 would suitably be adjusted to correspond to an airflow threshold which is exceedable by a user's inhalation.

Thus, in order to administer a dose, the user inhales creating a sufficient airflow to raise the flap 60 against the biasing force. This is illustrated in FIG. 3. As the flap 60 is raised by the airflow and pivoted around the second axle 68 (clockwise in FIG. 3), the mating portion of the flap 60, being on the other side of the axle is lowered, whereby the second end portion 66 of the prop 58 loses its support. This will cause the prop 58 to pivot around the first axle 64 (anticlockwise in FIG. 3) and to “roll” off the mating portion of the flap 60. The latch 56 is now in its second position, in which it allows the actuator 32 to move to said unloaded position. Thus, the stored energy of the coil spring 46 will cause the now released actuator 32 to move. The actuator 32 will pivot around its axle 36 and the jaws 34 will be raised, whereby the engaged separating element 20 is lifted from the base 14. The foil portion 18 remains attached to the separating element 20, thus opening the medicament-containing cavity 16. FIG. 1 illustrates with dashed lines a separating element 20 being raised by the jaws 34 of the actuator 32.

The latch 56 and the opening mechanism with its actuator 32 are comprised in what may generally be referred to as the dispensing mechanism. Thus, when the latch 56 has latched the actuator 32 in the energized position, the dispensing mechanism is in its primed state. When the user has inhaled so that the latch 56 has released the actuator 32 which has thereby moved to the unloaded position, the dispensing mechanism is in its fired state. As will be explained later, a disabler 102 is provided for preventing the dispensing mechanism to reach its primed state, e.g. by preventing the latch 56 from latching the actuator 32 in the energized position.

It is realized that the design of the exemplified inhaler 2 provides for use of a phenomenon denoted as shear driven cavity principle during deaggregation of the powder in the cavity 16 and emptying of the powder therefrom. The shear driven cavity is a model for flow in a cavity where the upper boundary moves in a desired flow direction, and thus causes a rotation in the cavity. FIG. 2 illustrates a medicament powder-containing cavity 16 having a suitable headspace above the powder. In FIG. 3, the inhalation airflow passes by said headspace along a flats surface region, said flat surface region comprising the opening into the powder-containing cavity 16. The horizontal passing of the inhalation airflow leads to a build-up of an eddy air stream in the cavity 16 which causes powder to be deaggregated and emptied from the cavity 16. The cavity 16 is generally brick-shaped and the cavity opening has a rim where the sides of the cavity transcend into the flow passage flat surface region. Accordingly, the airflow, when passing the cavity in the flow passage, preferably flows in parallel with a plane coinciding with the rim of the cavity opening in the flow passage.

While the flap 60 may return to the lowered position after a dose is dispensed, the jaws 34 of the actuator 32 will remain in the unloaded position (see e.g. FIG. 7) until the user primes the inhaler for the next dose.

Although the priming of the inhaler 2 may be coupled to either the opening or closing of the outlet cover 12, in this example embodiment, it is assumed that closing of the outlet cover 12 primes the inhaler 2. Thus, when the user has inhaled a dose (FIGS. 3 and 7), he/she will close the outlet cover 12 to cover the mouthpiece 10 (FIG. 1). Although, the io outlet cover 12 may be designed to form various travel paths, such as linear or stepwise paths, in this example embodiment the outlet cover 12 is rotated to cover the mouthpiece 10. During such closing of the outlet cover 12, the connected insert 38 with its force transmitting projecting member 50 and cam 44 will cause the jaws 34 of the actuator 32 to be lowered against the force of the coil spring 46 (FIG. 5) and the base 14 to is be rotated, thus presenting an unopened next cavity 16 to the jaws 34. The insert 38 will also press the position-keeping element 72 of the prop 58, causing the latch 56 to return to its first position, whereby the actuator 32 is prevented from lifting its jaws 34. After that, when the user opens the outlet cover 12 in order to take another dose, the insert 38 will rotate the other way without affecting the latched and energized actuator 32. The inhaler 2 is now primed (triggered) and ready to be fired when the user breaths in through the mouthpiece 10, thereby enabling breath-triggered lifting of a foil portion 18 from a cavity 16.

In order to reduce the risk of latching the actuator 32 in the energized position without having aligned an unopened cavity 16, the latch 56 is prevented from returning to the first latching position before the next cavity is aligned with the mouthpiece 10. Also in order to reduce the risk of overindexing, i.e. passing an unopened cavity 16 past the mouthpiece 10 without opening the cavity 16, an indexing mechanism for sequentially aligning the cavities with the mouthpiece 10 is provided, wherein the indexing mechanism is adapted to align the next cavity 16 with the mouthpiece 10 after the actuator 32 has been moved from the unloaded position to the energized position.

Thus, in the illustrated example embodiment, after a dose has been dispensed, the user closes the outlet cover 12. As has been described above, the rotation of the outlet cover 12 causes the generally disk-shaped insert 38 to rotate. Through the rotation of the insert 38, the provided cam 44 will urge the actuator 32 (see FIG. 5) to move to its energized position. Thus, the jaws 34 of the actuator 32 will move from the raised unloaded position illustrated in FIGS. 3 and 7 to the lowered energized position illustrated in FIGS. 2 and 6.

Substantially simultaneously with the cam 44 urging the actuator 32, through the rotation of the insert 38, the projecting second force transmitting member 50 will urge the indexing mechanism to advance the next cavity 16 to be aligned with the mouthpiece 10. More particularly, as illustrated in FIG. 6, the projecting member 50 will energize the torsion spring 52 which is connected to the drive member 54 (see FIG. 8). The energized torsion spring 52 will urge the connected drive member 54 to rotate around the central axis provided by the post 48 (see FIG. 1) in order to engage the base 14 and to thereby cause the base 14 to rotate so as to bring the next cavity 16 aligned with the mouthpiece 10.

However, the force on the drive member 54 provided by the projecting member 50 via the torsion spring 52 is temporarily counteracted, at least until the actuator 32 has reached its energized position. If the jaws 34 of the actuator 32 would not be lowered before indexing, the separating element 20 next in turn would risk hitting the jaws 34 during the indexing.

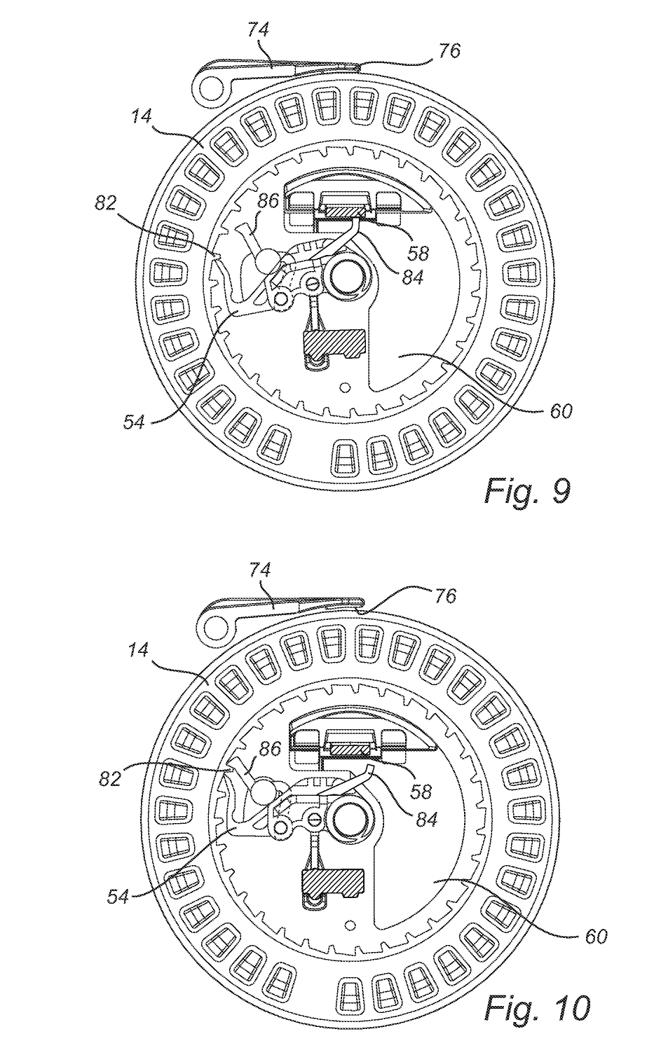

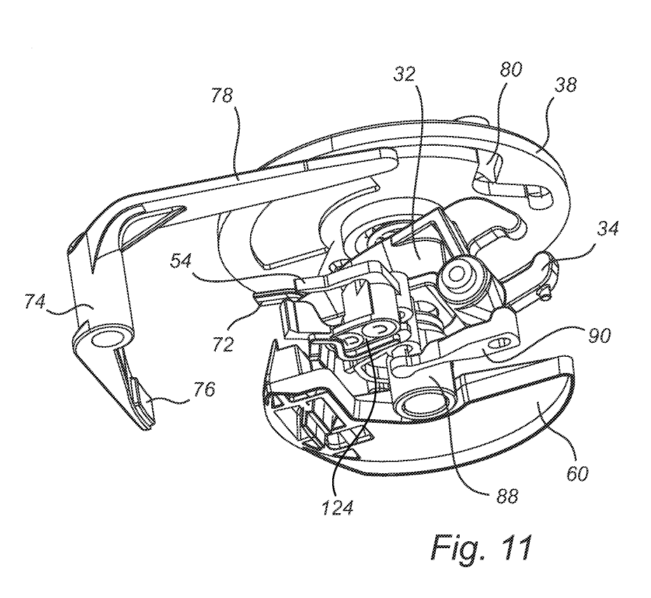

The counteracting member comprises a brake 74 adapted to prevent the compartments from moving. The brake 74 is attached to a lateral post 75 projecting from the lower housing portion 8 (see FIG. 1). The brake comprises a brake pad 76 which is pressed against the outer enveloping surface of the base 14 (see FIG. 9), thereby preventing the base 14 from rotating. The counteracting member also comprises a follower 78 (see FIGS. 1 and 11) which is connected to the brake 74 and which travels in a track 80 provided in the underside of the generally disk-shaped insert 38. The track 80 is best seen in FIGS. 4, 5 and 11, wherein FIG. 11 demonstrates how the follower 78 travels in the track 80. Thus, as the follower 78 travels in the track 80, it will follow an irregular path and when it reaches a point of release, the connected brake 74 lets go of the base 14 (FIG. 10). Now, the base 14 is allowed to be rotated by the drive member 54 which is urged by the torsion spring 52 as previously explained. Thus, the above exemplified mechanical sequencing assembly provides for alternate energizing of the opening mechanism (herein exemplified as the jawed actuator 32) and indexing of the compartments (herein exemplified as sealed cavities 16 in a base 14).

As illustrated in FIG. 9, before the brake 74 is released an end portion of the drive member 54 engages one of a plurality of teeth 82 in the base 14. An arm-shaped catch 84 is connected to the drive member 54 and may even be formed in one piece with the drive member 54. The catch 84 is in a preventing position, in which it prevents the first element (prop 58) of the latch 56 from becoming supported by the second element (flap 60) of the latch 56. Thus, in this state of the inhaler, the actuator cannot become latched in the energized position. Thus, the risk of re-firing from the same cavity 16 is reduced.

As the brake 74 is released, the drive member 54 will via the engaged tooth 82 rotate the base 14 one cavity-step. FIGS. 9 and 10 also illustrate a pawl 86 being pivotally mounted at a pivot point of the drive member (indicated with dashed lines). In FIG. 9, the is pawl 86 is retracted, while in FIG. 10 the pawl 86 has been advanced to engage with a tooth 82, herein illustrated as engaged with the opposite side of the same tooth 82 that is pushed by the drive member 54. The pawl 86 prevents the drive member 54 from over-rotating the base 14, ensuring that the inhaler is indexed only one cavity-step at a time.

The drive member 54 and the catch 84 are connected to a common barrel 88 (best seen in FIG. 11) which swivels around the central post 48 (FIG. 1) projecting upwardly from the lower housing portion 8. As the drive member 54 rotates the base 14 the catch 84 will be removed from the preventing position, as illustrated in FIG. 10, thereby allowing the prop 58 to become supported by the flap 60 and latch the energized actuator. The inhaler is now primed.

As previously described, in particular in connection with FIGS. 2 and 3, when the user opens the outlet cover 12 and inhales through the mouthpiece 10, the flap 60 is raised so that the prop 58 comes off the flap 60, thereby unlatching the actuator 32. The actuator 32 being energized by the coil spring 46 will be raised so that the jaws 34 of the actuator 32 remove the separating element 20 and the foil portion 18 from the cavity 16 presently aligned with the mouthpiece 10. As can be seen in FIG. 11, a movable pulling arm 90 connects the drive member 54 with the actuator 32. As the actuator 32 and the jaws 34 are raised, the pulling arm 90 follows that motion whereby at the other end of the pulling arm 90, the drive member 54 will be pulled from the primed state shown in FIG. 10 to the fired state shown in FIG. 9. The catch 84 will consequently be moved back to its preventing position shown in FIG. 9. Next, when the user closes the outlet cover 12, the inhaler will once again become primed.

If the user, for some reason, does not close the outlet cover 12 enough, the follower 78 travelling in the track 80 will not reach its point of release, and consequently the brake 74 will not be released. This in turn means that there will be no indexing. Furthermore, although the actuator 32 is in its energized position, it will not become latched, as latching can only occur in connection with indexing, as explained above. Thus, if the user then opens the outlet cover 12, which has not been fully closed, the actuator 32 will simply move back to its unloaded position.

The herein discussed indexing mechanism, enables rotation of the base 14 to be is limited to one direction. Thus, un-indexing may be prevented from occurring. This may be advantageous in connection with other types of opening mechanisms or separating elements.

When all the compartments have been opened, the next time the indexing mechanism rotates the base 14, a disabler 102 will be activated which is comprised in the disabler assembly 100. The disabler assembly 100 also comprises a tolerance take-up component 104. The tolerance take-up component has a circular portion 106 which fits within the inner enveloping surface of the annular base 14. The tolerance take-up component 104 comprises a mounting pin 108 which extends through a mounting hole 110 of the disabler 102 and is attached to a mounting sleeve 112 projecting from the lower housing portion 8. The disabler 102 comprises a spring element 114 which ends with said mounting hole 110. The disabler 102 is hereby, via the spring element 114, weakly anchored to the tolerance take-up component 104 to avoid uncontrolled movement of the disabler 102. The disabler 102 has a circular portion 116 which substantially matches the circular portion 106 of the tolerance take-up component 104.

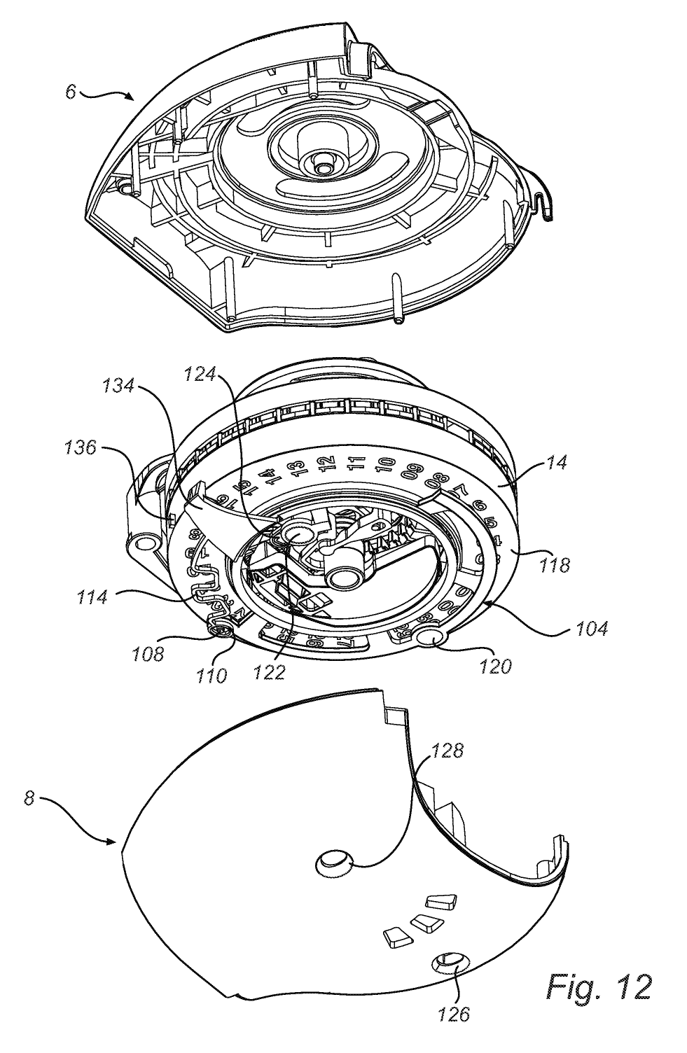

As can be seen in FIGS. 12, 13a ,14a and 15a, there is provided a dose count indicator 118, herein presented in the form of an annular foil being attached to the bottom of the base 14 and being provided with numbers 00, 01, 02, 03 . . . 28, 29, 30. In the illustrated example, it is assumed that the number of doses remaining to be dispensed will be presented to the user, although an alternative would be to present the number of doses that have alread been dispensed. The tolerance take-up component 104 comprises a first window 120, which is arranged to be aligned with the numbers on the dose count indicator 118 for presenting the number of doses remaining to be dispensed from the inhaler. The tolerance take-up component 104 also comprises a second window 122, which is arranged to be aligned with a status indicator 124 for presenting whether or not the inhaler is ready for inhalation, i.e. whether or not the dispensing mechanism is in the primed state. For simplicity, the status indicator 124 is herein shown with two symbols, one represents a cup with a lid (indicating that an unopened dose is available, i.e. primed state), and one represents an empty cup (indicating that the dose has been taken and is no longer available, i.e. fired state). Of course, any other suitable symbols, texts, colours, etc. may be used for representing the status of the inhaler. Both the first window 120 and the second window 122 may suitably comprise a magnifying glass. The first window 120 and the second window 122 are aligned with respective corresponding openings 126, 128 in the lower housing portion 8 (see FIG. 12). Suitably, in use, only one of the windows is visible at a time through the lower housing portion 8. For instance, when the oulet cover 12 is closed the first window 120 presenting the dose count is visible, and when the outlet cover 12 is open only the second window 122 presenting the status (ready or not ready) is visible.

In the embodiment illustrated in the drawings, the status indicator 124 is rigidly connected to the drive member 54 (see FIG. 11). Thus, when the drive member 54 performs the indexing action, i.e. moving to the position illustrated in FIG. 10, the connected status indicator 124 will also be moved. More particularly, the status indicator 124, will be moved so that the symbol representing an unopened cavity (status is “ready”) will be in register with the covering second window 122, e.g. as illustrated in FIG. 13a. As will be explained further below, when the last dose has been taken and the disabler 102 is activated, the status indicator 124 will be prevented from showing the “ready” status and will instead show the symbol representing “not ready”.

The disabler 102 also comprises a bevel or chamfer 130 which is adapted to come into contact with and push an abutment portion 132 of the flap 60 (see FIGS. 13b, 14b and 15b). Furthermore, the disabler 102 comprises a radially projecting portion 134 which extends from the circular portion 116 of the disabler 102 at the inner enveloping surface of the annular base 14 and across the base 14 to the outer enveloping surface of the base 14. The spring element 114 starts out from and is formed in one piece with the radially projecting portion 134. All parts of the disabler 102 may suitably be formed in one piece. An activator, herein illustrated in the form of a peg 136 on the enveloping surface of the base 14, is rotatable with the base 14 and adapted to come into contact with and push the radially projecting portion 134 of the disabler 102.

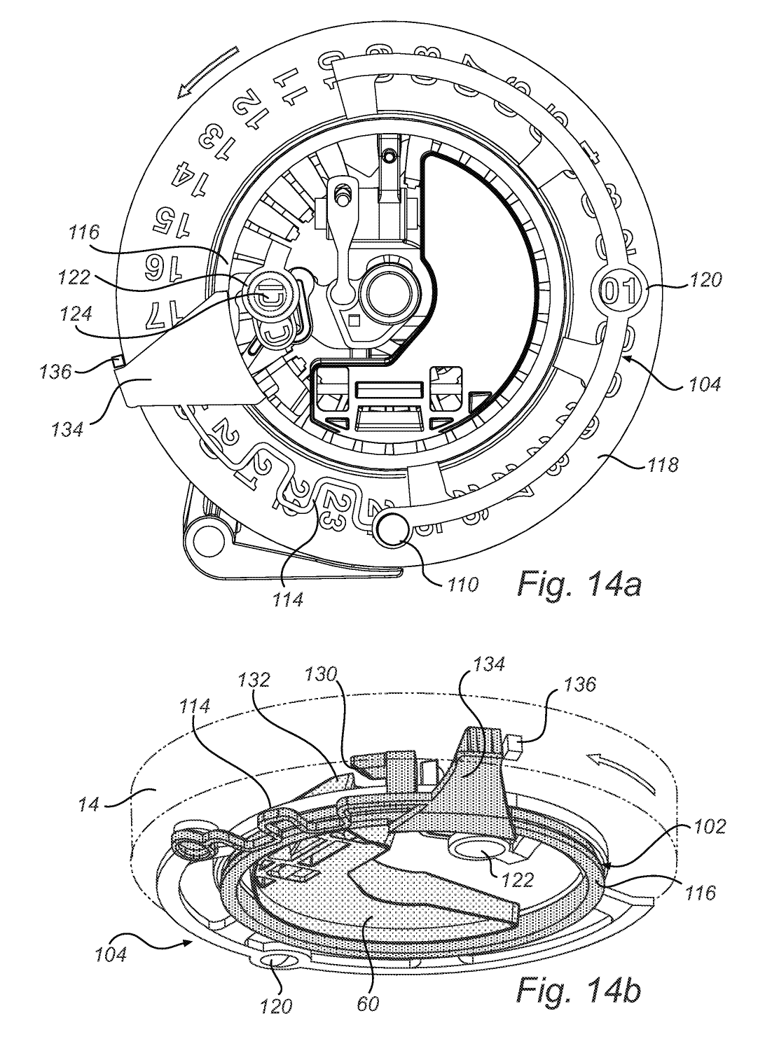

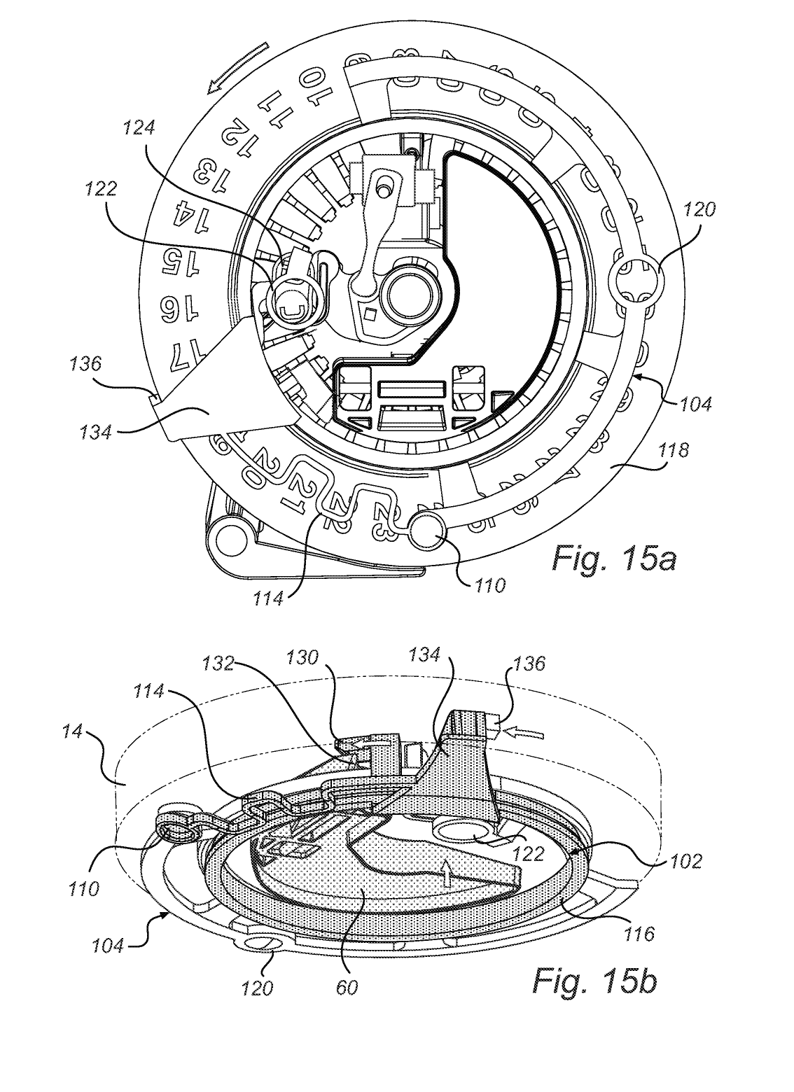

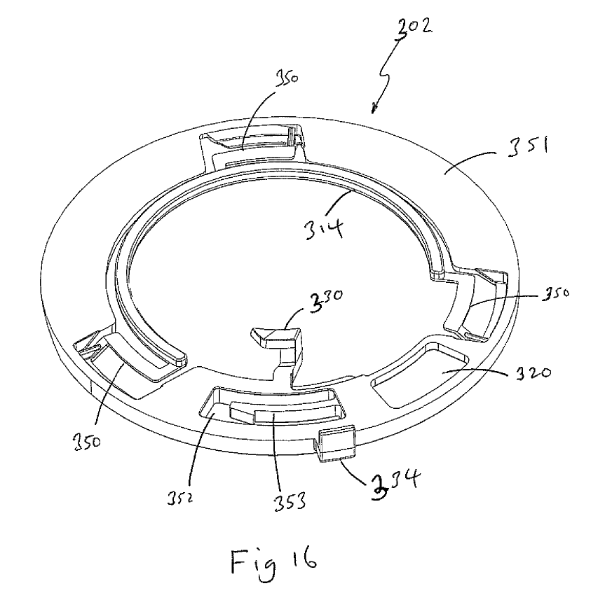

In FIG. 13a the dose count through the first window 120 shows that there are thirty doses remaining to be dispensed. Therefore, the peg 136 is thirty rotational steps (away is from pushing the radially projecting portion 134 of the disabler 102 (see also the perspective view in FIG. 13b). According to the representation in FIG. 13a, the peg 136 will be rotated stepwise in an anti-clockwise direction. FIGS. 14a and 14b illustrate the situation when only one dose remains to be dispensed. The dose count window 120 shows the number “01” and the status indicator window 122 shows an unopened cavity, i.e. “ready” for dispensing. At this point, the peg 136 has been rotated twenty-nine steps anti-clockwise and has reached a position next to (just behind) the radially projecting portion 134 of the disabler 102. When the last of the remaining thirty doses has been dispensed, and the indexing mechanism once again rotates the base 14 as has been discussed in more detail above, the peg 136 will push the radially projecting portion 134 of the disabler 102 (see FIGS. 15a and 15b). This will cause the radially projecting portion 134 to move towards the fixed mounting hole 110, thereby compressing the spring element 114. As a result of this rotational movement of the radially projecting portion 134, the chamfer 130 will come into contact with and push the abutment portion 132 of the flap 60. As the abutment portion 132 of the flap 60 is pushed, the flap 60 will be pivoted around its axle 68 and be somewhat raised, similarly to the illustration in FIG. 3. Thus, when the flap 60 is lifted, the latch 56 which incorporates the flap 60 will now be in its second position in which it does not latch the actuator 32 in the energized position, as has been previously discussed above in more detail. Since the actuator 32 can no longer become latched in the energized position when the outlet cover 12 is closed , it will automatically spring back to the unloaded position when the user opens the outlet cover 12 in order to inhale. Since the actuator 32 cannot remain in the energized position with the outlet cover 12 open, the dispensing mechanism which incorporates the actuator 32 cannot reach its primed state. Thus, despite an inhalation effort by the user, there will be no moving of the dispensing mechanism from a primed state to a fired state, and consequently the sound which has io previously been generated during such movement will not be generated this time. As previously explained, a movable pulling arm 90 connects the drive member 54 with the actuator 32 (FIG. 11). As the actuator 32 and the jaws 34 automatically spring back after the disabler 102 has been activated, the pulling arm 90 follows that motion whereby at the other end of the pulling arm 90, the drive member 54 will be pulled from the primed state is shown in FIG. 10 to the fired state shown in FIG. 9. This pull-back of the drive member 54 results in the connected status indicator 124 also becoming pulled back. Therefore, through the second window 122, the status indicator 124 will now show a “not ready” symbol to the user (see FIG. 15a). Thus, the activating of the disabler 102 has prevented the status indicator 124 from showing the “ready” symbol. Also illustrated in FIG. 15a, the indexing movement of the base 14 results in that behind the first window 120 the dose count number “01” will be replaced by “00”.

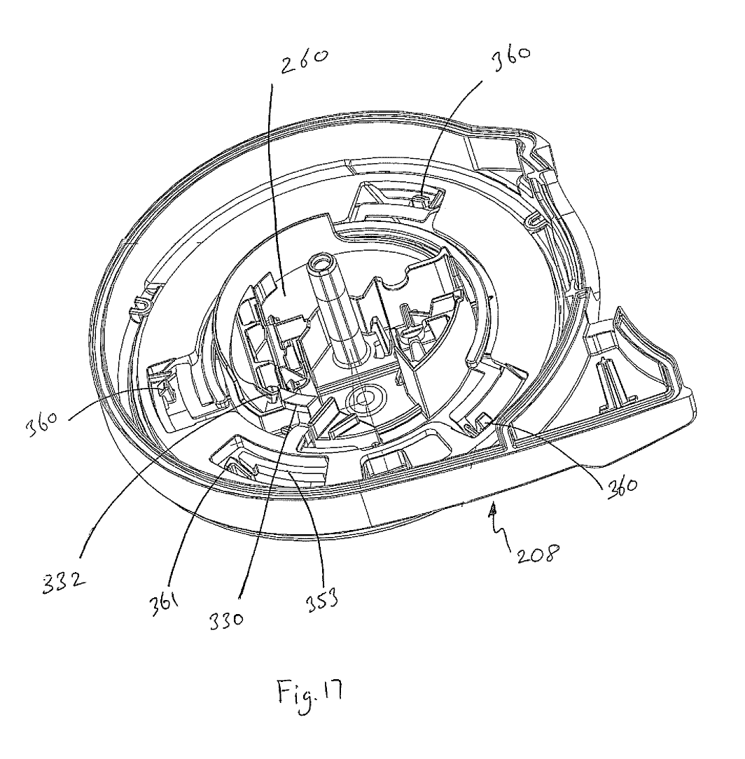

Referring now to FIGS. 16 to 18, a second embodiment is shown. Most of the components of this embodiment are similar to those of the previous embodiment and only the components relating directly to the “end of life” function will be described in detail.

In this embodiment, the design of the disabler has been changed and it now performs the functions of both the disabler 102 and tolerance take up member 104 in the previous embodiment.

The combined disabler 302 is best seen in FIG. 16. It is a flat ring structure of acetal material. A bevelled blocking member 330, corresponding to the bevel 120 in e.g. FIG. 14b, projects from a thin web 314 extending around the inner circumference of the ring. The web 314 is joined to the main disc 351 via joining webs 350 which are designed to allow for a degree of resilient deformation. The main disc 351 is formed with an approximately rectangular window aperture 320 and a further aperture in which is a barbed projection 353. On the outer periphery of the disc 351 is an outwardly radially projecting portion 334 whose function corresponds to that of the projection 134 in the previous embodiment.

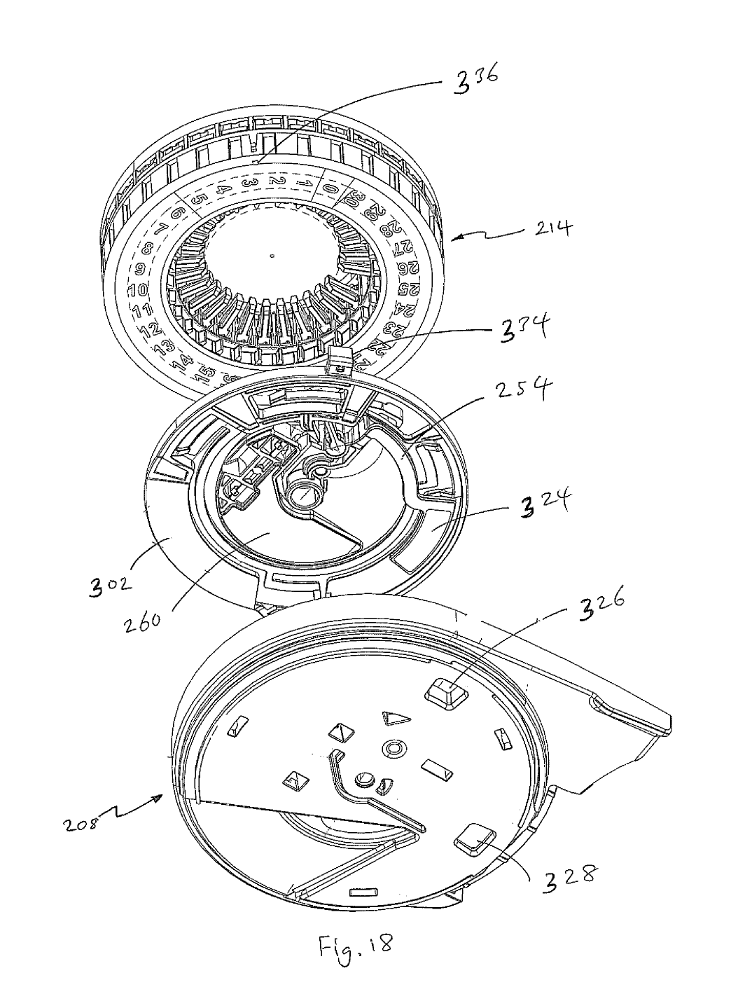

FIG. 17 shows the combined disabler 302 located in the lower housing portion 208. The disabler 302 is retained by snap fastenings 360 on the inner surface of the lower housing portion 208, in such as way as to allow limited rotational movement. FIG. 17 also shows the breath flap 260 with an abutment portion 332 against which the blocking member 330 will bear in the end of life condition, in a similar way to the interaction between the bevel 130 and abutment portion 132 in the previous embodiment. A locking lug 361 is provided on the inner surface of the housing, whose function will be exaplined later.

FIG. 18 is an exploded view showing a base 214, equivalent to base 14 of the previous embodiment, and its associated assembly. It also shows the disabler 302 and breath flap 260, together with a status indicator 324 corresponding in function to the status indicator 124 of the previous embodiment and being integrally moulded with an indexer component. FIG. 18 also shows the lower housing portion 208, which has window apertures 326, 328 for displaying the dose count and status indicator symbol, respectively.

The operation of this embodiment is very similar to that of the previous embodiment. As the final dose is used, a peg 336 on the base 214 (corresponding to the peg 136 of the previous embodiment) interacts with the projection 334 on the disabler 302. The disabler 302 is moved around on its snap fastenings 360 with respect to the lower housing portion 208, in a clockwise direction as viewed in FIGS. 16-18. The blocking member 330 engages with the abutment portion 332 of the breath flap 260, thereby blocking movement of the breath flap and preventing it from being latched. The barbed projection 353 is forced past the locking lug 361 (best seen in FIG. 17) and locks there, preventing any possibility of the disabler disc 302 moving back.

In a similar way to the previous embodiment, the status indicator, which is part of the indexer component, will show an empty cup (or similar symbol indicating no dose is available) through the window 328 in the housing.

It should be noted that in this application terms such as “upper”, “lower”, “above”, “below” have been used for explanatory purposes to describe the internal relationship between elements of the inhaler, regardless of how the inhaler is oriented in the surrounding environment. For instance, in the exemplified embodiment in the drawings, the cavities 16 are regarded as being placed “below” the foil portions 18, while the separating elements 20 are regarded as being placed “above” the foil portions 18, regardless of how the inhaler 2 as a whole is held or turned by the user. Similarly, “horizontal” means a direction located in the plane of the foil portions 18 or any plane parallel to the plane of the foil portions 18, and “vertical” means any direction perpendicular to such planes. Thus, a vertical line may intersect the cavities 16, the foil portion 18 and the separating elements 20.

Most components of the inhaler 2, such as the base 14, the separating elements 20, the actuator 32, the latch 56 and the disabler assembly 100 are suitably made of a plastic material, such as a polymer, however, other materials, such as metal or ceramic are conceivable alternatives.

The inhaler 2 may suitably comprise a structure that provides a moisture protection, such as e.g. a moisture absorbent sink as described in WO2006/000758, or any other appropriate alternative for including desiccant material.

It should be noted that although the drawings have been illustrated in connection with a dry powder inhaler having a disk with sealed cavities, the inventive concept encompasses and may be applied to other types of inhalers as well. Thus, disabling of the sound may be implemented in devices with strips carrying compartments, or blister packs, or any other form of dose carrying structure which can be indexed. Consequently, the inventive concept is not limited to disabling a sound of the herein illustrated separating elment 20 hitting the spring element 26 or some other portion of the guide structure 22, or a sound generated by the actual spring-induced movement of the actuator 32, but may very well be used with other types of opening mechanisms, such as mechanisms which pierce or punch through the compartments to enable access to the medicament.

1. An inhaler, comprising:
an outlet,
a plurality of sealed compartments configured to contain doses of medicament to be dispensed through said outlet;
a dispensing mechanism having a primed state, wherein the dispensing mechanism is ready for dispensing a dose, and a fired state, wherein the dispensing mechanism has dispensed a dose, wherein the dispensing mechanism moves from the primed state to the fired state in response to an inhalation flow, wherein the dispensing mechanism generates a sound when the dispensing mechanism moves from the primed state to the fired state; and
a disabler which, after the final dose has been dispensed, is configured to disable the dispensing mechanism from generating said sound.

2. The inhaler of claim 1, wherein the disabler is configured to prevent the dispensing mechanism from reaching the primed state.

3. The inhaler of claim 1, wherein the dispensing mechanism further comprises:
an opening mechanism having an energized position in which the opening mechanism is biased towards an unloaded position, wherein, during movement from the energized position to the unloaded position, the opening mechanism is configured to open a sealed compartment aligned with the outlet; and
a latch having a first position, in which the latch is configured to latch the opening mechanism in the energized position, and a second position, in which the latch is configured to allow the opening mechanism to be in said unloaded position, wherein the latch is at least partly arranged in a flow path such that an inhalation flow through the flow path is configured to move the latch from the first to the second position,
wherein, when the disabler is activated, the disabler is configured to prevent the latch from reaching the first position.

4. The inhaler of claim 3, wherein the latch includes a pivotable element for changing between the first and second positions of the latch, wherein, when activated, the disabler is configured to pivot the latch away from the first position in order to prevent the opening mechanism from becoming latched.

5. The inhaler of claim 1, further comprising an outlet cover movable for alternatingly closing and opening the outlet, the outlet cover being operatively connectable to the dispensing mechanism in order to, upon one of said closing or opening movements of the outlet cover, move the dispensing mechanism to the primed state, wherein, when the disabler is activated, the dispensing mechanism is prevented from reaching the primed state despite said movements of the outlet cover.

6. The inhaler of claim 3, further comprising:
a base having said plurality of sealed compartments and including an activator; and
an indexing mechanism configured to move the base in order to sequentially align the compartments with the outlet,
wherein, when the indexing mechanism indexes the base after the final dose has been dispensed, the activator is configured to activate the disabler to disable the dispensing mechanism from generating said sound.

7. The inhaler of claim 6, further comprising;
a status indicator for indicating to the user whether the inhaler is ready or not ready, the status indicator being connected to the indexing mechanism to follow motions of the indexing mechanism, wherein, when the indexing mechanism indexes the base, the status indicator changes an indication from not ready to ready,
wherein, when the disabler disables the dispensing mechanism, the status indicator is prevented from maintaining the ready indication.

8. The inhaler of claim 7,
wherein the indexing means is configured to index the base after the opening mechanism has been moved from the unloaded position to the energized position,
wherein, when the disabler prevents the latch from latching the opening mechanism in the energized position, the opening mechanism is enabled to return to the unloaded position,
wherein the opening mechanism is connected to the indexing mechanism in such manner that when the opening mechanism moves from the energized position to the unloaded position, the indexing mechanism is set in reverse motion, and the status indicator changes from ready to not ready.

9. The inhaler of claim 6, wherein the disabler has a disabling position, in which the disabler is configured to prevent the dispensing mechanism from generating said sound, and a removed position, in which the disabler is configured to allow the dispensing mechanism to generate said sound, wherein the activator is mounted on the base and configured to move the disabler to the disabling position.

10. The inhaler of claim 7, wherein said base includes a rotatable disk having a circumferentially-oriented sequence of cavities, each cavity being sealed by a respective foil portion.

11. The inhaler of claim 10, wherein each foil portion is attached to a respective separating element, wherein, upon rotation of the disk, the separating element next in turn is presented to the opening mechanism for removal of the separating element and the attached foil portion.

12. The inhaler of claim 10, wherein the activator is configured to rotate with the disk and move one rotational step for each indexing of the base.

13. The inhaler of claim 1, wherein the medicament comprises an active ingredient including one or more of mometasone; ipratropium bromide; tiotropium and salts thereof; salemeterol; fluticasone propionate; beclomethasone dipropionate; reproterol; clenbuterol; rofleponide and salts; nedocromil; sodium cromoglycate; flunisolide; budesonide; formoterol fumarate dihydrate; terbutaline; terbutaline sulphate; salbutamol base and sulphate; fenoterol; 3-[2-(4-Hydroxy-2-oxo-3H-1,3-benzothiazol-7-yl)ethylamino]-N[-2[2-(4-methylphenyl)ethoxy]ethyl]propane-sulphonamide; hydrochloride; indacaterol; aclidinium bromide; N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g., dihydrobromide); N-Cyclohexyl-N³-[2-(3-fluorophenyl)ethyl]-N-(2-{[2-(4-hydroxy-2-oxo-2,3-dihydro-1,3-benzothiazol -7-yl)ethyl]amino}ethyl)-β-alaninamide or a pharmaceutically acceptable salt thereof (e.g., di-D-mandelate); a [2-(4-Chloro-benzyloxy)-ethyl]-[2-((R)-cyclohexyl-hydroxy -phenyl-methyl)-oxazol-5-ylmethyl]-dimethyl-ammonium salt (e.g., hemi-naphthalene-1,5-disulfonate); and a (R)-1-[2-(4-Fluoro-phenyl)-ethyl]-3-((S)-2-phenyl-2- piperidin-1-yl-propionyloxy)-1-azonia-bicyclo[2.2.2]octane salt (e.g., bromide or toluenesulfonate).

14. An inhaler, comprising;
an outlet;
a display for presenting a status indicator to a user, the status indicator having a first indicia representing a ready to inhale status of the inhaler and a second indicia representing a not ready to inhale status of the inhaler;
an outlet cover movable between a first position in which the outlet cover is configured to cover the outlet and a second position in which the outlet is uncovered, wherein, when said outlet cover is in the first position, the status indicator is out of view to the user, and wherein, when said outlet cover is in the second position, the status indicator is visible in said display;
a dispensing mechanism having a primed state, wherein the dispensing mechanism is ready for dispensing a dose, and a fired state, wherein the dispensing mechanism has dispensed a dose, wherein, when the dispensing mechanism is in the primed state, the first indicia of the status indicator is aligned with the display, and when the dispensing mechanism is in the fired state, the second indicia of the status indicator is aligned with the display; and
a disabler which, after the final dose has been dispensed, is configured to prevent the dispensing mechanism from being in said primed state when the outlet cover is in its second position, which allows the display to present the second indicia of the status indicator to the user.

15. (canceled)
